(19) United States
(12) Patent Application Publication
(10) Pub. No.: US 2026/0272309 A1
(43) Pub. Date: Sep. 17, 2026

(54) WEARABLE DEVICES FOR MULTI-MODAL MONITORING OF A MEDICAL CONDITION

(71) Applicant: Analog Devices, Inc., Wilmington, MA (US)

(72) Inventors: Shreeya Sriram, Woburn, MA (US); Ajit Sharma, Sudbury, MA (US); Vasundhara Sandip Langade, Burlington, MA (US); Tony J. Akl, Bedford, MA (US); Venugopal Gopinathan, Boston, MA (US)

(21) Appl. No.: 19/562,275

(22) Filed: Mar. 10, 2026

Related U.S. Application Data
(60) Provisional application No. 63/770,797, filed on Mar. 12, 2025.

(51) Int. Cl. A61B 5/0205; A61B 5/00; A61B 5/257; A61B 5/282; A61B 7/04; A61B 90/00

(52) U.S. Cl. CPC A61B 5/0205; A61B 5/257; A61B 5/282; A61B 5/6823; A61B 5/6833; A61B 7/04; A61B 90/03; A61B 2090/033; A61B 2562/0204; A61B 2562/0219; A61B 2562/028; A61B 2562/164

(57) ABSTRACT

A wearable device comprising a flexible frame for mounting to a subject's torso in accordance with a defined placement. The frame comprises a first elongated section (“first section”) having a first end and a second elongated section (“second section”) having a second end. The sections are spaced apart. Each section has a pair of electrodes. A member extends from a portion of the first section and includes a sensor device configured to generate measurement data representative of chest sounds. In the defined placement, the first end is positioned adjacent the subject's sternal centerline, the first section extends towards the subject's left midaxillary line, the second section extends along the midaxillary line, and the member is positioned adjacent the subject's sternum and at least a portion of the member covers an area within a thoracic cavity of the subject, with the sensor device abutting at least a portion of the area.

### CROSS-REFERENCE TO RELATED APPLICATIONS

This application claims the benefit of U.S. Provisional Application No. 63/770,797, filed on Mar. 12, 2025 and entitled “WEARABLE DEVICES FOR MULTI-MODAL MONITORING OF A MEDICAL CONDITION,” the contents of which are incorporated by reference herein in the entirety.

### TECHNICAL FIELD

The present disclosure relates generally to wearable devices, and more specifically, to a wearable device for multi-modal monitoring of a medical condition.

### BACKGROUND

Some medical conditions, such as a cardiopulmonary condition, are progressively debilitating and may require significant medical intervention even when treated. Monitoring a medical condition of a subject can reduce the need for or avoid medical intervention.

Conventional wearable devices for monitoring a medical condition of a subject may contact skin that overlays the sternum of the subject. Such contact can cause discomfort for the subject, especially if the subject has had a surgical procedures or other types of procedure that cause scar tissue on the skin that overlays the sternum. Discomfort associated with conventional wearable devices may reduce adherence to a self-administered monitoring protocol. As a result, if the subject does not adhere to a self-administered monitoring protocol, the condition may have progressively deteriorated and/or may have been inadequately treated for a prolonged period of time prior to the subject visiting a clinic or a clinician office. This can have dire consequences for the subject, which may in some cases result in costly medical interventions and unfavorable prognosis.

Additionally, some conventional wearable devices may be difficult to place and/or wear for subjects having a substantive amount of breast tissue or with higher body mass index (BMI). Placement of some conventional wearable devices for monitoring cardiopulmonary conditions may be challenging, potentially reducing adherence to a self-administered monitoring protocol in subjects with limited dexterity. Some conventional wearable devices also require multiple steps for placement and/or require sizing to a particular subject before use, adding complexity to the monitoring process. Therefore, improvements are desired.

### SUMMARY

This summary is provided to introduce a selection of concepts in a simplified form that are further described below in the DETAILED DESCRIPTION. This summary is not intended to identify key features of the claimed subject matter, nor is it intended to be used as an aid in determining the scope of the claimed subject matter.

Aspects of the present disclosure include a wearable device for multi-modal monitoring of a medical condition. The wearable device comprises a flexible frame configured to be mounted to a torso of a subject in accordance with a defined placement. The flexible frame comprises a first elongated section having a first end and including a first pair of electrodes. In the defined placement, the first end is positioned adjacent a sternal centerline of the subject, and the first elongated section extends towards a left midaxillary line of the subject. The flexible frame further comprises a second elongated section having a second end and including a second pair of electrodes. The second elongated section is spaced apart from the first elongated section. In the defined placement, the second elongated section extends along the left midaxillary line. The wearable device further comprises an extension member integrated with the flexible frame. The extension member extends from a portion of the first elongated section and includes a sensor device configured to generate measurement data representative of chest sounds, In the defined placement, the extension member is positioned adjacent a sternum of the subject and at least a portion of the extension member covers an area within a thoracic cavity of the subject, with the sensor device abutting at least a portion of the area.

Aspects of the present disclosure include a wearable device for multi-modal monitoring of a medical condition. The wearable device comprises a flexible frame configured to be mounted to a subject according to a defined placement. The flexible frame comprises a first elongated section having a first end and including a first pair of electrodes and an inertial sensor device configured to generate first measurement data representative of first chest sounds. In the defined placement, the first end is positioned adjacent a sternal centerline of the subject and the first elongated section extends towards a left midaxillary line of the subject. The flexible frame further comprises a second elongated section having a second end and including a second pair of electrodes and a second inertial sensor device configured to generate second measurement data representative of second chest sounds. The second elongated section is spaced apart from the first elongated section. In the defined placement, the second elongated section extends along the left midaxillary line.

Aspects of the present disclosure include a system for multi-modal monitoring of a medical condition. The system comprises a flexible frame configured to be mounted to a torso of a subject in accordance with a defined placement. The flexible frame comprises a first elongated section and a second elongated section spaced apart from the first elongated section. Each elongated section has a corresponding end and includes a corresponding pair of electrodes. The system further comprises at least one sensor device attached to the flexible frame, one or more memories storing instructions therein, and one or more processors communicatively coupled with the one or more memories. The one or more processors are configured, individually or in any combination, to initiate a measurement of thoracic impedance of the subject via the at least one sensor device, receive, from the at least one sensor device, measurement data representative of chest sounds, and exchange one or more measurement signals based on the measurement data with another device.

### BRIEF DESCRIPTION OF THE DRAWINGS

The accompanying drawings form part of the disclosure and are incorporated into the subject specification. The drawings illustrate example aspects of the disclosure and, in conjunction with the following detailed description, serve to explain at least in part various principles, features, or aspects of the disclosure. Some aspects of the disclosure are described more fully below with reference to the accompanying drawings. However, various aspects of the disclosure is implemented in many different forms and should not be construed as limited to the implementations set forth herein. Like numbers refer to like elements throughout.

FIG. 1 is a schematic block diagram of an example system to monitor health status of a subject, in accordance with one or more aspects of this disclosure.

FIG. 2 is a schematic diagram of an example portion of an upper body of a subject, including the torso of the subject, illustrating the ribcage and heart of the subject, in accordance with one or more aspects of this disclosure.

FIG. 3A is a plan view of an example device to monitor health of a subject using multiple measurement modalities, in accordance with one or more aspects of this disclosure.

FIG. 3B is another plan view of the example device illustrated in FIG. 3A, opposite the plan view shown in FIG. 3A, in accordance with one or more aspects of this disclosure.

FIG. 4 is a schematic diagram of an example portion of an upper body of a subject, including the torso of the subject, in accordance with one or more aspects of this disclosure.

FIG. 5A is a plan view of another example device to monitor health of a subject using multiple measurement modalities, in accordance with one or more aspects of this disclosure.

FIG. 5B is another plan view of the example device illustrated in FIG. 5A, opposite the plan view shown in FIG. 5A, in accordance with one or more aspects of this disclosure.

FIG. 6A is a plan view of another example device to monitor health of a subject using multiple measurement modalities, in accordance with one or more aspects of this disclosure.

FIG. 6B is another plan view of the example device illustrated in FIG. 6A, opposite the plan view shown in FIG. 6A, in accordance with one or more aspects of this disclosure.

FIG. 7 is a schematic block diagram of an example processing platform to monitor health status of a subject, in accordance with one or more aspects of this disclosure.

FIG. 8 is a schematic block diagram of another example system to monitor health status of a subject, in accordance with one or more aspects of this disclosure.

FIG. 9 is a schematic block diagram of yet another example system to monitor health status of a subject, in accordance with one or more aspects of this disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known components may be shown in block diagram form in order to avoid obscuring such concepts.

Embodiments of this disclosure enable monitoring a status of a medical condition of a subject. The medical condition may be a cardiopulmonary condition of the subject. A cardiopulmonary condition includes, for example, chronic obstructive pulmonary disease (COPD) and/or congestive heart failure (CHF). Even when treated, a cardiopulmonary condition is progressively debilitating and can cause the subject to undergo significant medical intervention. Thus, monitoring the status of one or more cardiopulmonary conditions of the subject can reduce the need for or avoid medical intervention.

Monitoring the status of a cardiopulmonary condition typically involves ambulatory care. As a result, an exacerbation of the cardiopulmonary condition may be identified during a visit to a clinic or a clinician office. Thus, in some cases, the condition may have progressively deteriorated and/or may have been inadequately treated for a prolonged period of time prior to the visit to the clinic or the clinician office. Having a prolonged deterioration of the cardiopulmonary condition or inadequately treating the condition for a prolonged period of time can have dire consequences for the subject, which may in some cases result in costly medical interventions and unfavorable prognosis. Further, subjects may have difficulty adhering to a long-term routine of ambulatory care to monitor the status of cardiopulmonary conditions, which can lead to identification of a status change after a prolonged deterioration of the cardiopulmonary condition or when the subject is in distress. Both situations being clearly undesirable and detrimental to favorable prognosis.

The present disclosure relates generally to wearable devices, and more specifically, to a wearable device for multi-modal monitoring of a medical condition. Aspects of the present disclosure described herein provide systems and wearable devices that, individually or in combination, permit monitoring a cardiopulmonary condition of a subject and/or contemporaneous identification of changes in the status of the cardiopulmonary condition. Such a monitoring is non-ambulatory (non-ambulatory-care monitoring). With non-ambulatory-care monitoring, the subject does not have to visit a health services facility (e.g., a clinic, an office of a clinician, etc.), and the monitoring is performable in a dwelling (e.g., a home, a group home, a nursing home, etc.) that is remotely located from health services facilities.

Some systems in accordance with this disclosure include a wearable device configured to be mounted to the torso of a subject. The wearable device operates according to one or a combination of multiple sensing modalities, and provides one or several types of measurement signals associated with a bodily function of the subject. The wearable device includes multiple electrodes and multiple sensor devices. Each one of the multiple electrodes is configured to contact a respective skin section of the torso of the subject. The multiple sensor devices include at least one electric sensor device functionally coupled (e.g., electrically coupled and/or mechanically coupled) with the multiple electrodes. Each electric sensor device measures a voltage, a current, an impedance, a combination thereof, or similar physical quantities. In some alternative or additional aspects, at least one electric sensor device supplies a current and/or applies a voltage. The current is an AC current or a DC current. The voltage is an AC voltage or a DC voltage.

In contrast to some conventional wearable devices for monitoring a medical condition of a subject, the wearable devices in accordance with aspects of the present disclosure reduce or avoid contact with skin that overlays the sternum of a subject. This provides improved comfort for subjects who have had surgical procedures, such as Coronary Artery Bypass Graft (CABG) or other types of procedures that cause scar tissue on the skin that overlays the sternum. By improving comfort, the wearable devices of the present disclosure promote adherence to a self-administered monitoring protocol.

The wearable devices in accordance with aspects of the present disclosure are configured to be worn on one side of the chest of a subject. In some aspects, the wearable devices in accordance with aspects of the present disclosure are configured to be worn on the left side of the chest of a subject, in proximity to the left clavicle and slightly off center so that a heavier section of the wearable devices' rests on the flat portion of the chest above the left breast of the subject. As a result, for subjects having a substantive amount of breast tissue—whether the subject is a biological woman, a transgender woman, or an individual with higher body mass index (BMI) —the wearable devices in accordance with the present disclosure provide superior comfort compared to conventional wearable devices, thereby promoting adherence to a self-administered monitoring protocol. Greater adherence to a monitoring protocol assists with timely identifying changes to the status of a cardiopulmonary condition and reducing or avoiding decompensation that might result in hospitalization or other substantial medical intervention.

In some aspects, a wearable device in accordance with aspects of the present disclosure comprises a flexible frame. In some alternative or additional aspects, the flexible frame is a monolithic structure having a defined non-adjustable size, providing a one-size-fits-all structure. As a result, the wearable device need not be sized to the subject being monitored, which eliminates the need for a clinician or another caregiver to size the wearable device to a particular subject before use. In some alternative or additional aspects, the flexible frame has attached thereto an adhesive layer that covers a planar surface thereof. In some aspects, a single non-adhesive removable layer overlays the adhesive layer and is removed before placement on the chest. In some alternative aspects, multiple non-adhesive removable layers overlay the adhesive layer and are removed before placement on the chest. By having an adhesive layer that is exposed entirely by removal of a single non-adhesive removable layer or multiple non-adhesive removable layers, placement of the wearable device is significantly simplified relative to conventional monitoring devices, which may improve adherence to a monitoring protocol in subjects with limited dexterity.

Placement of the wearable device on an upper portion of the chest of a subject—a section that is generally flat irrespective of biological sex of the subject and body size—makes placement easier relative to placement of some conventional wearable devices for monitoring cardiopulmonary conditions. Ease of placement may improve adherence to a monitoring protocol in subjects with limited dexterity.

Multi-modal monitoring enabled by wearable devices in accordance with aspects of the present disclosure is the use of multiple sensing modalities, such as electrical sensing, acoustic sensing, and inertial sensing, to generate measurement signals representative of different physiological functions of a subject. By employing multiple sensing modalities, the wearable devices and systems of the present disclosure can contemporaneously acquire thoracic impedance data, electrocardiogram signals, chest sounds, and chest vibrations, thereby providing a more comprehensive assessment of a cardiopulmonary condition than single-modality devices.

In some aspects, each electric sensor device is configured to generate measurement signals during a measurement period. The measurement period spans several pulmonary ventilation cycles (i.e., breathing cycles). A first measurement signal of the measurement signals includes a thoracic impedance (TI) measurement signal. In some aspects, the TI measurement signal is generated at a defined sampling rate. In one non-limiting example, the TI measurement signal is generated in a four-point (or four-electrode) measurement configuration and at a defined sampling rate of 40 KHz. In some alternative or additional aspects, a second measurement signal of the measurement signals includes an electrocardiogram (ECG) signal, i.e., electric potential changes between a pair of electrodes configuring as a lead. Each electric sensor device generates two or more ECG signals corresponding to respective leads. An ECG signal is sampled at either the same sampling rate that the TI measurement signal is sampled at or a different sampling rate.

In some alternative or additional aspects, the multiple sensor devices include one or more acoustic sensor devices configured to generate other measurement signals during the measurement period. The acoustic sensor devices, individually or in any combination, probe sounds originating in or propagating through an organ that is present within the chest cavity and/or adjacent areas of the subject. In one example configuration, at least one first acoustic sensor device of the acoustic sensor devices serves as at least one lung auscultation probe. In another example configuration, the at least one first acoustic sensor device serves as at least one heart auscultation probe. In some alternative or additional aspects, at least one second acoustic sensor device of the acoustic sensor devices serve as at least one heart auscultation probe.

In some alternative or additional aspects, the multiple sensor devices include an inertial sensor device configured to generate other measurement signals during the measurement period. In one non-limiting example, the inertial sensor device is a three-axis accelerometer. The measurement signals are used to generate electrical signals corresponding to movement of the chest wall of the subject and/or vibrations from the chest cavity during the measurement period.

In some alternative or additional aspects, the multiple sensor devices include an optical device that permits measuring oxygen saturation level, and generates a measurement signal accordingly. In one non-limiting example, the optical device is a pulse oximeter. In some alternative or additional aspects, the multiple sensor devices include a blood pressure meter. It is noted that the technology described herein is not limited to the sensor devices described herein.

In some alternative or additional aspects, systems in accordance with this disclosure include one or more computing devices. In some alternative or additional aspects, the computing devices are deployed as a processing platform. In some alternative or additional aspects, the computing devices are server devices arranged in a cloud architecture. In one non-limiting example, at least one of the server devices operates as a gateway device, at least one of the server devices operates as a computer node, and/or at least one of the server devices operates as a storage node.

In some alternative or additional aspects, the processing platform is communicatively coupled with the wearable device via one or more networks (e.g., one or more wireless networks, one or more wired networks, or a combination of both). The processing platform, via the networks and at least one of the computing devices, receives measurement signals generated by the wearable device over time. The processing platform, via at least one of the computing devices, implements numerous processes based on the received measurement signals. At least some of the processes include determining values of a particular metric or a combination of metrics based on the measurement signals. In some aspects, the particular metric is indicative of status of the cardiopulmonary condition of the subject. In some alternative or additional aspects, the combination of metrics is indicative of the status of the cardiopulmonary condition of the subject. Non-exhaustive examples of the metrics include respiratory rate (RR), relative tidal volume (TV), ratio of an exhalation time and an inhalation time of the subject, delay between mechanical onset of inspiration, and onset of inspiration airflow. Respiratory rate indicates the frequency of breathing cycles. Relative tidal volume indicates the amount of air exchanged during breathing. The ratio of exhalation time to inhalation time provides insight into airway obstruction. The delay between mechanical onset of inspiration and onset of inspiration airflow may indicate respiratory muscle dysfunction, fluid buildup, or airway resistance.

In some aspects, a metric is a quantity directly probed by measurement signals generated by a sensor device. In some alternative or additional aspects, a non-limiting example metric is a blood oxygen level directly measured by an oximeter present in the wearable device. Aspects of the present disclosure permit tracking temporal changes of one or more metrics such as, but not limited to, at least one of respiratory rate, relative tidal volume, ratio of exhalation time and inhalation time, and delay between mechanical onset of inspiration and onset of inspiration airflow. By monitoring one or more metrics individually and combinations of two or more metrics, the systems provide detection and differentiation of cardiopulmonary exacerbations.

In some alternative or additional aspects, at least some of the processes, when implemented, permit tracking temporal changes of multiple metrics. The processes include determining multiple values of a particular metric or a combination of metrics over a time interval. The time interval spans two or more measurement intervals, each of the measurement intervals resulting in a value of each one of the metrics being tracked. In some alternative or additional aspects, the processes include monitoring, over the time interval, using the multiple values, a time-dependence of a particular metric or a combination of metrics. In some alternative or additional aspects, monitoring such a time-dependence includes updating parameters defining a function (linear or otherwise) that fits currently determined values of the particular metric or the combination of metrics. In some alternative or additional aspects, monitoring such a time-dependence includes updating a parameter defining a rolling average of currently determined values of the particular metric or the combination of metrics. Regardless of how the time-dependence is monitored, the time-dependence of the particular metric or the combination or metrics conveys a trend (e.g., an upward trend, a downward trend, or an essentially constant trend). By monitoring one or more metrics individually and/or combinations of two or more metrics, aspects of the present disclosure described herein provide detection and differentiation of cardiopulmonary exacerbations.

In some alternative or additional aspects, systems in accordance with the present disclosure include a computing device that is remotely located relative to the processing platform. The computing device is communicatively coupled with the processing platform via one or more networks. In some aspects, such network are the same networks that permit communicatively coupling the wearable device with the processing platform described herein. In some alternative or additional aspects, the computing device is associated with, or otherwise operated by, a clinician and/or an insurance provider. The clinician is involved in providing care for a cardiopulmonary condition of a subject. As such, the computing device receives information related to a status of the cardiopulmonary condition of the subject and/or a change in the status of the cardiopulmonary condition of the subject. In some alternative or additional aspects, at least a portion of the information is formatted as an electronic message that is presented by a software application, or another type of program code, configured to execute in the computing device. The computing device, via the software application, receives and presents the information.

In some alternative or additional aspects, the computing device is communicatively coupled with a user device via one or more networks. In some aspects, such networks are the same networks that permit communicatively coupling with the computing device with the processing platform described herein. In some alternative or additional aspects, the user device is part of one or more of the systems in accordance with the present disclosure. The user device is associated with, and operated by, the subject wearing the wearable device and/or by an aid (e.g., a family member, a nurse assistant, or nursing home staff) assisting the subject with performing measurements. The user device is configured with a software application, or another type of program code, to exchange information with the computing device. As such, the computing device, via the software application configured in the computing device, provides an indication of an action to be executed by the subject in response to a particular value of a metric being monitored and/or a status change in the cardiopulmonary condition. The type of action is associated with the nature of the status change.

In some alternative or additional aspects, the user device is communicatively coupled with the wearable device. The user device is communicatively coupled with the processing platform via one or more networks. In some aspects, such networks are the same networks that permit communicatively coupling the wearable device with the processing platform described herein.

In some alternative or additional aspects, the software application, or another type of program code, which is configured in the user device controls the manner of performing measurements using the wearable device. In response to the software application being executed, the user device presents a user interface that permits configuring a measurement session, e.g., a measurement period, number of measurements to be performed during the measurement period, a combination thereof, or similar attributes of the measurement session. The user device presents one or more other user interfaces that display various types of information, including observed data (e.g., ECG data, thoracic impedance, peripheral oxygen saturation (SpO₂), etc.) from the measurements.

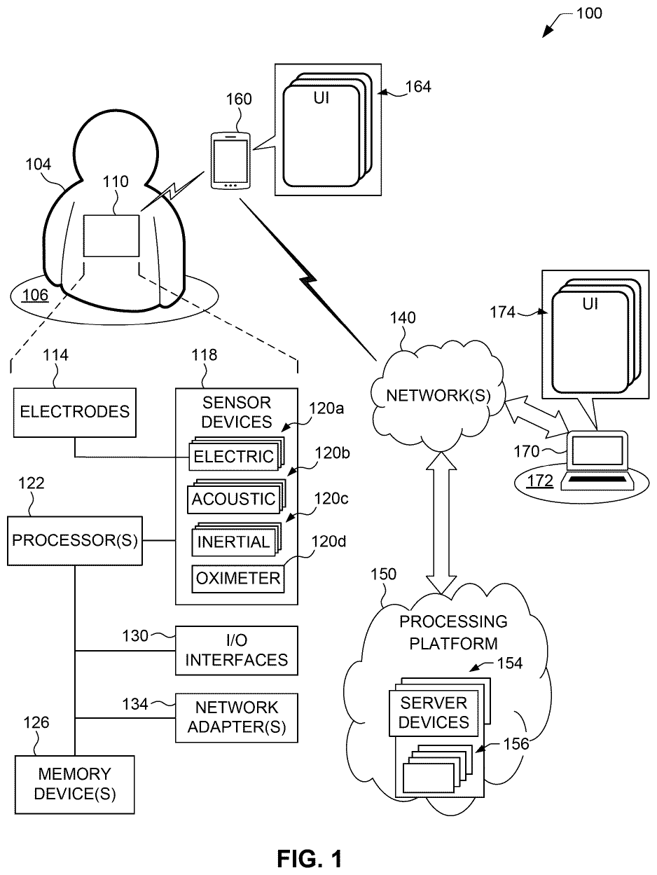

FIG. 1 is a schematic block diagram of an example system 100 for monitoring status of a cardiopulmonary condition of a subject, in accordance with aspects of this disclosure. The example system 100 includes a wearable device 110 configured to be mounted to the torso of a subject 104. For example, in some aspects, the wearable device 110 is mounted to the chest of the subject 104. In some aspects, the subject 104 is located in a dwelling 106 or another type of facility. The wearable device 110 operates according to a single sensing modality or a combination of multiple sensing modalities. The wearable device 110 provides one type of measurement signal or several types of measurement signals associated, individually or in combination, with a bodily function of the subject 104. In one non-limiting example, the bodily function is a respiration function and/or a cardiac function. The wearable device 110 includes multiple electrodes 114 and one or more sensor devices 118. Each one of the multiple electrodes 114 is configured to contact a respective skin section of the chest of the subject 104.

In some aspects, the sensor devices 118 include one or more electric sensor devices 120a coupled with the multiple electrodes 114. Each electric sensor device 120a is configured to measure a voltage, a current, an impedance, a combination thereof, or similar physical quantities. In some alternative or additional aspects, at least one of the electric sensor devices 120a is configured to supply a current and/or apply a voltage. The current that is supplied is a DC current or an AC current. The voltage that is applied is a DC voltage or an AC voltage. In some alternative or additional aspects, each electric sensor device 120a is configured to generate a measurement signal during a measurement period. In some aspects, the measurement signal is an electrical measurement signal. Electric measurement signals generated over time are used to monitor the cardiopulmonary condition by probing pulmonary ventilation characteristics and/or cardiac characteristics of the subject 104. In some alternative or additional aspects, the measurement period spans several pulmonary ventilation cycles (i.e., breathing cycles), each cycle extending between initiation of inhalation and termination of exhalation. As a non-limiting example, the measurement period has a duration within a range from about 15 seconds(s) to about 120 s. Example durations of the measurement period include 25 s, 30, s, 45 s, 60 s, 70 s, 80 s, 90 s, 100 s, 110 s, and 120 s.

In some aspects, a first electric sensor device 120a of the group of electric sensor devices 120a measures impedance. The first electric sensor device 120a is configured to generate measurement signals indicative or otherwise representative of impedance. In some aspects, as the wearable device 110 is mounted to the torso of the subject 104, the impedance that the first electric sensor device 120a measures is thoracic impedance. Accordingly, the measurement signals includes thoracic impedance (TI) measurement signals. In some alternative or additional aspects, the TI measurement signals are generated in a four-point configuration (or four-electrode measurement) or in a two-point configuration (or two-electrode measurement). A TI measurement signal generated over a measurement period is representative of an amount of air that is present in the lungs of the subject 104 as the subject 104 breathes during the measurement period, i.e., the temporal dependence of TI measurement signal represents the amount of air present in the lungs as a function of time. Accordingly, measurements of thoracic impedance that are performed by the wearable device 110 substitute the need for direct measurements of the amount of air that is present in the lungs during breathing.

As measurements of thoracic impedance are performed where the subject 104 is located, such measurements permit non-ambulatory-care monitoring of pulmonary ventilation characteristics of the subject 104 without reliance on a health services facility to access dedicated equipment (such as spirometry equipment) and without subjecting the subject 104 to direct measurements of the volume of air that enters and exits the lungs of the subject 104. As a result, in sharp contrast to conventional technologies and ambulatory-care monitoring, the non-ambulatory-care monitoring the system 100 enables provides a wealth of pulmonary ventilation data with a time resolution that is superior to that of commonplace ambulatory-care monitoring.

In some alternative or additional aspects, a second electric sensor device 120a of the group of electric sensor devices 120a generates measurement data representative of electrical activity in the heart of the subject 104. The second electric sensor device 120a measures voltage differences corresponding to respective particular combinations of two or more electrodes 114 of the multiple electrodes 114. The particular combinations of two or more electrodes 114 are configured as respective leads, where each of the leads defines an ECG vector. The leads correspond or are similar to at least a subset of standard precordial leads (V1, V2, V3, V4, V5, V6) or standard limb leads (Lead I, Lead II, Lead III, aVF, aVL, and aVR). In some alternative or additional aspects, a first electrode 114 and a second electrode 114 of the multiple electrodes 114 are configured as a lead that is similar to the standard limb lead Lead II. Accordingly, the second electric sensor device 120a generates measurement signals representative of electrical activity that permits detecting P-waves in an ECG of the subject 104. Configuring electrodes as a lead similar to Lead II permits generation of measurement data representative of electrical activity that allows detection of P-waves in an ECG of the subject, as well as detection of other morphological features of ECG signals. Detection of P-waves is clinically significant for identifying atrial arrhythmias that may be associated with cardiopulmonary conditions such as CHF.

In some alternative or additional aspects, the sensor devices 118 include one or more acoustic sensor devices 120b. Each acoustic sensor device 120b includes a piezoelectric microphone or a microelectromechanical system (MEMS) microphone, and is configured to generate measurement signals during a measurement period. The acoustic sensor devices 120b, individually or in any combination, probes chest sounds originating in or propagating through a lung and/or the heart present within the chest cavity of the subject 104. In one example, at least one first acoustic sensor device 120b of the acoustic sensor devices 120b serve as a respective lung auscultation probe by generating measurement signals representative of lung sounds. In some alternative or additional aspects, at least one second acoustic sensor device 120b of the acoustic sensor devices 120b serves as a respective heart auscultation probe by generating measurement signals representative of heart sounds.

In some alternative or additional aspects, the sensor devices 118 include one or more inertial sensor devices 120c. The wearable device 110 is mounted to the subject 104 such that each inertial sensor device 120c is mechanically coupled to the chest of the subject 104. Specifically, each inertial sensor device 120c is in contact with a solid medium (e.g., a garment) that, in turn, is in contact with the chest of the subject 104. Accordingly, each inertial sensor device 120c generates an inertial measurement signal representative of chest vibrations. Chest vibrations include contributions from motion caused by various physiological phenomena. In some alternative or additional aspects, these contributions include a first type of contributions and a second type of contributions. The first type of contributions originate from movement of the chest wall, circulation of blood outside the heart of the subject 104, and/or other physiological phenomena besides lung sounds and heart sounds. The second type of contributions originate from heart sounds or lung sounds, or both.

In some alternative or additional aspects, the one or more inertial sensor devices 120c consists of a single inertial sensor device 120c. As a non-limiting example, the single inertial sensor device 120c includes a multiple-axis (e.g., three-axis) accelerometer device. In some alternative or additional aspects, the one or more inertial sensor devices 120c include multiple inertial sensor devices 120c, e.g., multiple-axis (e.g., three-axis) accelerometer devices and/or multiple-axis (e.g., three-axis) gyroscope devices.

In some alternative or additional aspects, where a first inertial sensor device 120c of the inertial sensor devices 120c is a three-axis accelerometer, the three-axis accelerometer generates three inertial measurement signals corresponding to three orthogonal axes, such as the axes x, y, and z of a Cartesian coordinate system. In some aspects, the z axis is oriented along the direction of gravity. The inertial measurements collectively represent (i) an orientation of the three-axis accelerometer (or the wearable device 110) relative to the chest wall of the subject 104, and (ii) a rectilinear acceleration vector.

In some alternative or additional aspects, the sensor devices 118 include an oximeter device 120d that permits measuring oxygen saturation level. The oximeter device 120d is configured to generate measurement signals during a measurement period. In some alternative or additional aspects, the sensor devices 118 include a blood pressure meter (not shown in FIG. 1).

It is noted that aspects of the present disclosure are not limited to the sensor devices 118 described herein. In some alternative or additional aspects, one electric sensor device 120a of the electric sensor devices 120a is used to measure a temperature of the subject 104.

The wearable device 110 is depicted as a single block mounted to the torso of the subject 104 in FIG. 1 simply for purposes of illustration. In some embodiments, the wearable device 110 is a monolithic structure.

In some alternative or additional aspects, the wearable device 110 includes one or more processors 122, one or more memory devices 126, one or more I/O interfaces 130, and one or more network adapters 134. At least one of the processors 122 and at least one of the memory devices 126 forms a control unit. In some aspects, the control unit implements control operations involved in setup/placement of the wearable device 110 and/or performance of one or more measurements by at least one of the sensor devices 118, resulting in measurement signals. In some alternative or additional aspects, implementing the control operation involved in setup/placement of the wearable device 110 causes the wearable device 110 to present visual cues and/or aural cues. These cues are presented to the subject 104 and/or to a caregiver involved in the care of the subject 104. In some alternative or additional aspects, a first network adapter of the network adapters 134 is embodied in a radio unit that permits exchanging information wirelessly between the wearable device 110 and another device (e.g., computing device 170, user device 160, and/or a server device 154). In some aspects, the information includes one or more measurements signals.

In some alternative or additional aspects, the wearable device 110 is communicatively coupled with a processing platform 150 that also is part of the example system 100. Such functional coupling is implemented in numerous ways, and permits the wearable device 110 to supply data and/or signaling (e.g., control instructions) to the processing platform 150. In some aspects, as is illustrated in FIG. 1, the wearable device 110 is communicatively coupled with the processing platform 150 via a user device 160 and one or more networks 140 (e.g., one or more wireless networks, one or more wired networks, or a combination of both). In some alternative or additional aspects, the wearable device 110 is communicatively coupled with the processing platform 150 via the networks 140, without the user device 160 serving an intermediary element that permits such coupling. In some alternative or additional aspects, the user device 160 is configured with a software application, or another type of program code, to exchange information with other devices and present information corresponding or otherwise associated with measurements of the various signals described herein, as performed using the wearable device 110. In some aspects, the user device 160 presents at least some of the information in one or more user interfaces 164.

In some alternative or additional aspects, the processing platform 150 includes multiple server devices 154. The processing platform 150, via the networks 140 and at least one first server device 154 of the server devices 154, receives measurement signals generated by the wearable device 110 over time. The processing platform 150, via at least one second server device 154 of the server devices 154, implements numerous processes that use the received measurement signals. Such processes permit determining status or status change of the cardiopulmonary condition of the subject 104. In some alternative or additional aspects, the at least one second server device 154 retains a group of modules 156 that, in response to being executed by processors, cause the at least one second server device 154, individually or in combination, to perform at least one of the processes. At least some of such processes include determining values of a particular metric or a combination of metrics based on the measurement signals. In some aspects, the particular metric is indicative of status of a cardiopulmonary condition of the subject 104. In some alternative or additional aspects, the combination of metrics is indicative of the status of the cardiopulmonary condition of the subject 104. In some aspects, the group of modules 156 are implemented as hardware (digital electronics, for example), as software, or a combination thereof.

In some alternative or additional aspects, the processing platform 150 evaluates various types of metrics that permit, individually or in combination, identifying a status change of a cardiopulmonary condition. A type of a metric is either direct type or aggregate type. A metric of the direct type (i.e., a direct metric) is a quantity that is directly probed by a sensor device 118, i.e., measurement signals generated by the sensor device 118 are indicative of respective values of the metric. The quantity is a physical quantity, a chemical quantity, or a physicochemical quantity. Non-limiting examples of the physical quantity include thoracic impedance and speed of sound propagation. A non-limiting example of the chemical quantity is oxygen saturation. In some alternative or additional aspects, the oximeter device 120d directly probes oxygen saturation level. A measurement signal generated by the oximeter device 120d is indicative of the oxygen saturation level. In some alternative or additional aspects, the second server devices 154 obtains an observed value indicating the oxygen saturation level from the measurement signal generated by the oximeter device 120d. The second server device 154 assigns the observed value to a direct metric corresponding to oxygen saturation.

A metric of the aggregate type (i.e., aggregate metric) is a quantity derived by operating on measurement signals generated by a sensor device 118. In some alternative or additional aspects, the second server device 154 operates on measurements signals received from the wearable device 110. The second server device 154 operates on measurement signals according to one or more processes corresponding to the aggregate metric being evaluated. Some aggregate metrics are derived by operating on one type of measurement signals, e.g., TI measurement signals. Other aggregate metrics are derived by operating on two or more types of measurement signals. Non-limiting examples of aggregate metrics that are derived by operating on TI measurement signals include RR, relative TV, and ratio of exhalation time and inhalation time. Non-limiting examples of aggregate metrics that are derived by operating on TI measurement signals and inertial measurement signals include a delay between mechanical onset of inspiration and onset of inspiration airflow.

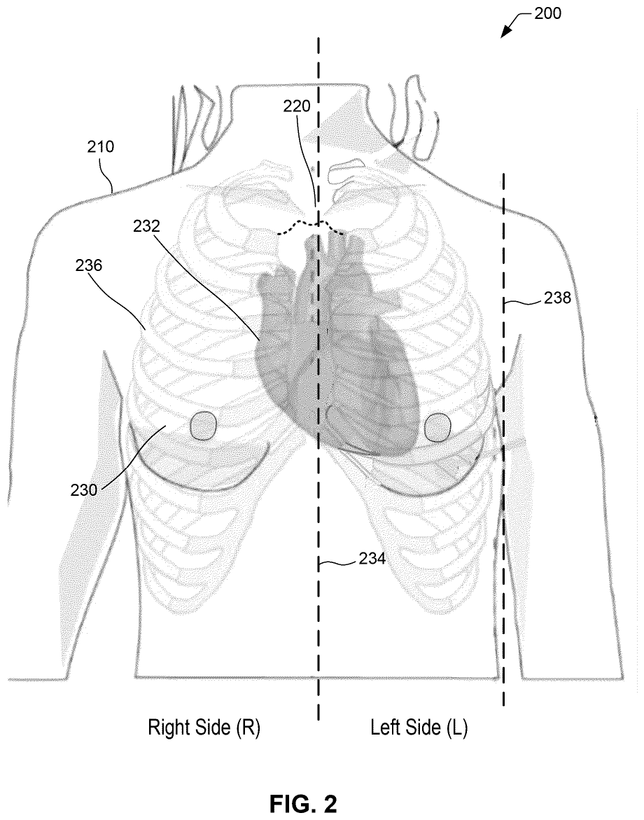

FIG. 2 is a schematic diagram 200 of an example part of the upper body of a subject 210, in accordance with one or more aspects of this disclosure. The schematic diagram 200 presents a frontal view of a part of the torso of the subject 210, illustrating a portion of the thoracic ribcage 236 and the heart 232 of the subject 210. The schematic diagram 200 also illustrates, with a dotted line, an edge of the sternum, to represent the suprasternal notch 220 of the subject 210. The schematic diagram 200 also partially outlines breast tissue 230 of the subject 210, where nipples are represented by circular areas. The schematic diagram 200 further illustrates the sternal centerline 234 and the left midaxillary line 238 of the subject 210. The sternal centerline 234 divides the torso of the subject 210 into a right side and a left side, which sides are denoted herein by “R” and “L,” respectively.

In contrast to some conventional wearable devices for monitoring a medical condition of a subject, the wearable devices in accordance with aspects of this disclosure reduce or avoid contact with skin that overlays the sternum of a subject. As a result, the wearable devices of this disclosure provide improved comfort for subjects who have had surgical procedures, such as CABG or other types of procedures that cause scar tissue on the skin that overlays the sternum. By improving comfort, the wearable devices of this disclosure promote adherence to a self-administered monitoring protocol.

Further, the wearable devices in accordance with aspects of this disclosure are configured (e.g., printed, molded, assembled, or otherwise manufactured) to be worn/placed on the left side of the chest of a subject, in proximity to the left clavicle of the subject and slightly off center so that a heavier section of the wearable devices rests on the flat portion of the chest above the left breast the subject. In some alternative or additional aspects, placement in proximity to the left clavicle is placement within about 2 cm to about 5 cm below the left clavicle. As a result, for subjects having a substantive amount of breast tissue—be the subject a biological woman, a transgender woman, or an individual with higher body mass index (BMI) —, the wearable devices in accordance with this disclosure provide superior comfort, thereby promoting adherence to a self-administered monitoring protocol. Greater adherence to a monitoring protocol assists with timely identifying changes to status of a cardiopulmonary condition and/or reducing or avoiding decompensation that might result in hospitalization or other substantial medical intervention.

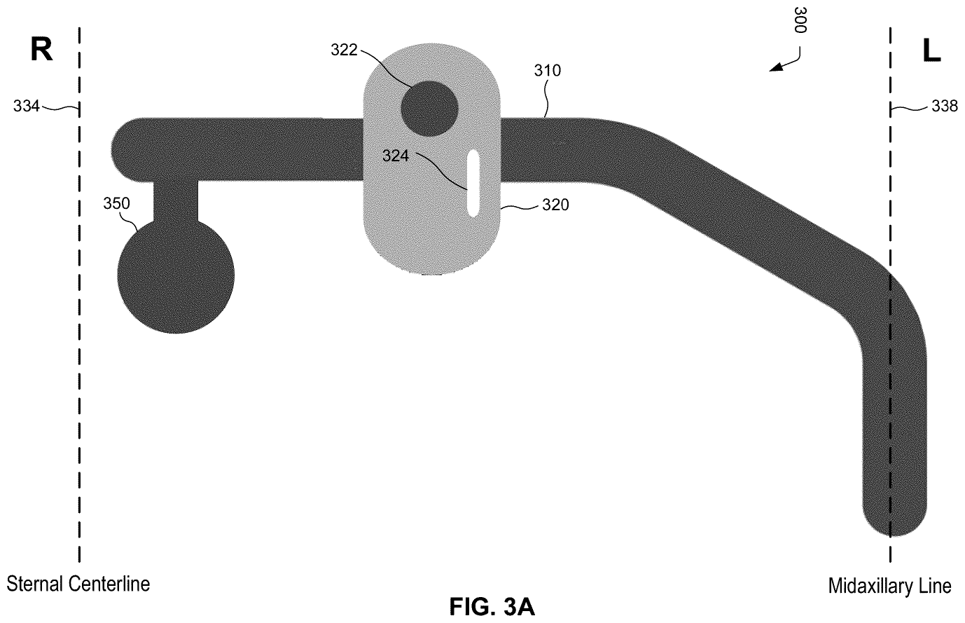

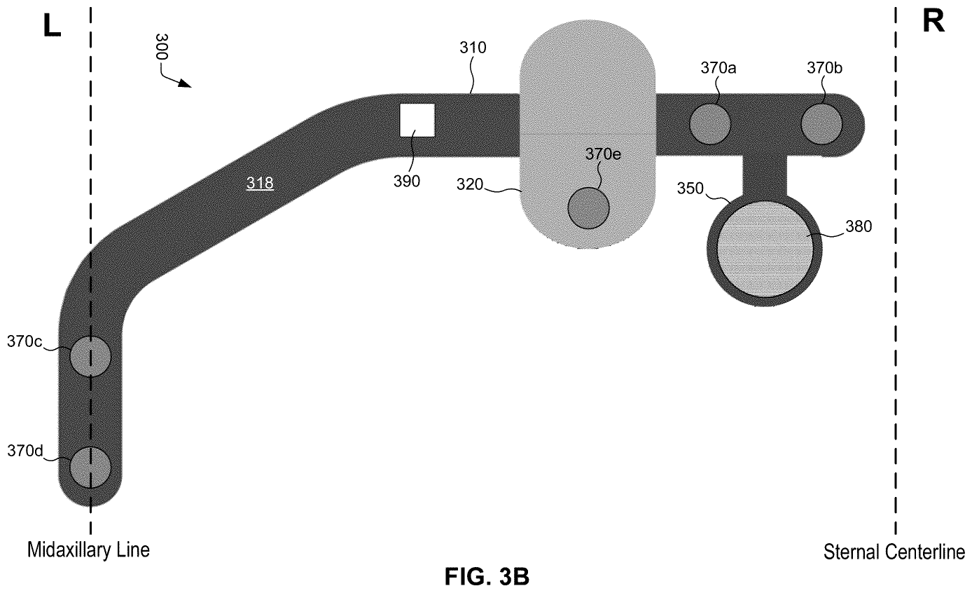

In FIGS. 3A-3B, 5A-5B, and 6A-6B, reference labels R and L denote right and left from the perspective of a subject whose torso a wearable device is mounted to.

FIG. 3A and FIG. 3B are respective plan views of an example wearable device 300 to monitor health of a subject using multiple measurement modalities, in accordance with one or more aspects of this disclosure. The plan view shown in FIG. 3A is a top view in that the plan view presents the example wearable device 300 as observed from a vantage point located over the chest of the subject and oriented towards an outer surface of the chest of the subject. The plan view shown in FIG. 3A presents the example wearable device 300 as viewed from outside the body of the subject, towards the chest of the subject. The other plan view shown in FIG. 3B is opposite the top view shown in FIG. 3A. Accordingly, the plan view shown in FIG. 3B is a bottom view in that the plan view presents the example wearable device 300 as viewed from a surface of the chest the subject, towards the exterior of the body of the subject. In some alternative or additional aspects, the bottom view is skin side, i.e., presents the side of a wearable device in accordance with this disclosure that contacts the skin of a subject wearing the wearable device (e.g., the example wearable device 300) in the prescribed placement described herein. In one non-limiting example, the example wearable device 300 embodies or forms part of the wearable device 110 (FIG. 1).

With reference to FIG. 3A, the example wearable device 300 includes a flexible frame 310. In some alternative or additional aspects, the example wearable device 300 further comprises a housing unit 320. The housing unit 320 contains one or more of the processors 122, memory devices 126, the I/O interfaces 130, the network adapters 134, or the electric sensor devices 120a. In some alternative or additional aspects, the housing unit 320 includes a button 322 or another interface element that energizes and/or initiates a measurement using the example wearable device 300. In some alternative or additional aspects, the housing unit 320 includes one or more light source devices, one or more speakers, and/or other types of I/O elements 324 that are part of the I/O interfaces 130. The flexible frame 310 is configured to be mounted to the chest of a subject according to a defined placement. In some alternative or additional aspects, the flexible frame 310 is a monolithic structure having a defined non-adjustable size, i.e., the flexible frame 310 is a one-size-fits-all structure. As a result, the example wearable device 300 need not be sized to the subject being monitored. For example, a clinician or another caregiver does not need to size the example wearable device 300 to a particular subject being monitored before the example wearable device 300 is used to monitor a medical condition of that particular subject.

The disclosure is not limited to the flexible frame 310 being a monolithic structure. In some alternative or additional aspects, the flexible frame 310 is assembled from various separate members having appropriate shape and size so as to provide a one-size-fits-all structure in accordance with aspects of this disclosure.

In the defined placement, the flexible frame 310 is placed on the left side of the chest of the subject, i.e., the flexible frame 310 is placed to the left of the sternal centerline 334 of the subject, spatially separated from the sternum (not depicted in FIG. 3A) of the subject, towards the left midaxillary line 338 of the subject. As such, in the defined placement, the flexible frame 310 reduces or avoids contact with skin that overlays the sternum. In some alternative or additional aspects, in the defined placement, the flexible frame 310 at least partially surrounds the breast tissue of the subject from above, i.e., from a region proximate the left clavicle of the subject, where the region is located on the subject. In the defined placement, the wearable device 300 is placed on the subject between the sternal centerline 334 and the left midaxillary line 338.

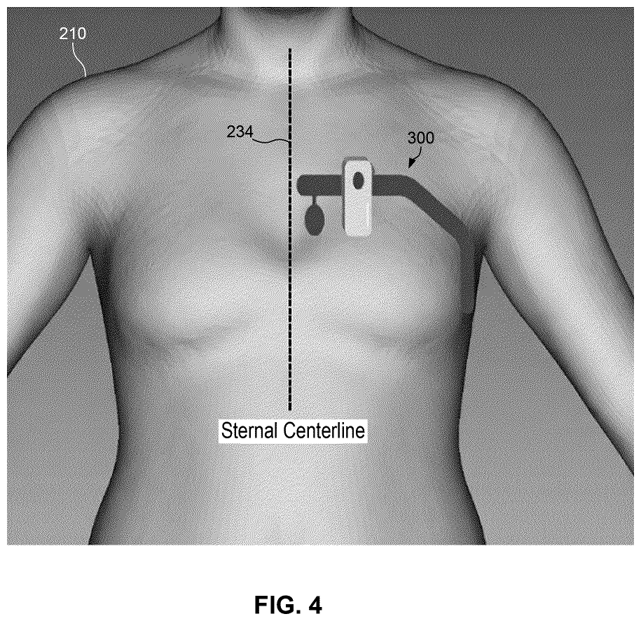

In one non-limiting example, the subject is the subject 210. FIG. 4 illustrates the defined placement of the example wearable device 300 on the torso of the subject 210.

Back to FIG. 3A, the flexible frame 310 is be elongated and extends from a first end to a second end along a path having a first bend and a second bend. More specifically, the flexible frame 310 includes a first elongated section having the first end, where in the defined placement, the first end is positioned adjacent the sternal centerline 334 and the first elongated section extends towards the left midaxillary line 338. The flexible frame 310 also includes a second elongated section having the second end, with the second elongated section being spaced apart from the first elongated section. In the defined placement, the second elongated section extends along the left midaxillary line 338.

In some alternative or additional aspects, the flexible frame 310 includes an extension member 350 integrated with the flexible frame 310. The extension member 350 extends from a portion of the first elongated section. In the defined placement, the extension member 350 is positioned adjacent the sternum (not depicted in FIG. 3A) of a subject, and at least a portion of the extension member 350 covers an area within a thoracic cavity (chest cavity) of the subject. In one non-limiting example, the portion of the extension member 350 covers an area on a left side of the thoracic cavity. In one non-limiting example, the portion of the extension member covers an area adjacent or over a heart of the subject that is within the thoracic cavity.

The example wearable device 300 further includes multiple electrodes. In some aspects, the wearable device 300 includes a first electrode 370a, a second electrode 370b, a third electrode 370c, and a fourth electrode 370d. In some alternative or additional aspects, the example wearable device 300 includes a fifth electrode 370e. As is illustrated in FIG. 3B, the first elongated section has integrated therein or attached thereto a first pair of electrodes consisting of the first electrode 370a and the second electrode 370b. Further, the second elongated section has integrated therein or otherwise attached thereto a second pair of electrodes consisting of the third electrode 370c and the fourth electrode 370d.

In some alternative or additional aspects, the first pair of electrodes and the second pair of electrodes are used for measurements of thoracic impedance in a four-point configuration. An electrode 370a/370b of the first pair of electrodes is configured as a first sense electrode and an electrode 370c/370d of the second pair of electrodes is configured as a second sense electrode. Another electrode 370b/370a of the first pair of electrodes is configured as a first force electrode, and another electrode 370d/370c of the second pair of electrodes is configured as a second force electrode. In some aspects, the first force electrode is configured to inject current into the chest of the torso of a subject (e.g., subject 210 (FIG. 2)) and the second force electrode is configured to receive the current injected into the chest of the subject, i.e., the first force electrode and the second force electrode force circulation of the injected current through the chest of the subject. In some alternative or additional aspects, the first force electrode is a positive terminal “F+” and the second force electrode is a negative terminal “F−.” The first sense electrode and the second sense electrode are configured to sense a voltage difference caused by the current injected into the chest of the subject. In some alternative or additional aspects, the first sense electrode is a positive terminal “S+” and the second sense electrode is a negative terminal “S−.” The measured thoracic impedance is based on the voltage difference and the amount of current injected into the chest of the subject. In some alternative or additional aspects, the first electrode 370a and the second electrode 370b are configured as the first sense electrode and the first force electrode, respectively. In some alternative or additional aspects, the third electrode 370c and the fourth electrode 370d are configured as the second sense electrode and the second force electrode.

In some alternative or additional aspects, in addition to measurement data indicative of thoracic impedance of a subject, the example wearable device 300 permits generating measurement data indicative of electrical activity of a subject, as part of generating an ECG. The fifth electrode 370e serves as a Right-Leg Drive (RLD) electrode and a pair of the first electrode 370a, the second electrode 370b, the third electrode 370c, or the fourth electrode 370d are configured as a limb lead that is similar to Lead II (one of the six limb leads in a standard 12-lead ECG). In one non-limiting example, one of the following combinations of electrodes are configured as Lead II: the first electrode 370a and the third electrode 370c; the first electrode 370a and the fourth electrode 370d; the second electrode 370b and the third electrode 370c; or the second electrode 370b and the fourth electrode 370d.

In some alternative or additional aspects, in the defined placement of the example wearable device 300 onto a chest of a subject (e.g., subject 210 (FIG. 2)), measurement data representative of electrical activity permits detecting P-waves in ECG. Consequently, the example wearable device 300 is used to monitor COPD of the subject, thus detecting COPD or trends in the health status of the subject that are indicative of the subject being at risk of COPD or progressing towards onset of COPD.

In some alternative or additional aspects, as is illustrated in FIG. 3B, the extension member 350 has integrated therein or attached thereto a sensor device 380 configured to generate measurement data representative of chest sounds. At least a portion of the extension member 350 covers an area within a thoracic cavity (chest cavity) of the subject with the sensor device 380 abutting at least a portion of the area. In some alternative or additional aspects, the sensor device 380 is a piezoelectric microphone, including a slab (disc-shaped or otherwise) of a lead zirconate titanate (PZT) crystal or another type of piezoelectric material. The piezoelectric microphone is of variable size (e.g., large or small). As a non-limiting example, a piezoelectric microphone that is large has a slab of piezoelectric material having a characteristic length (e.g., base or diameter) in a range from about 43 mm to about 53 mm. Regardless of its size, in the defined placement described herein, the piezoelectric microphone is placed on an upper portion of the chest of a subject. Such a section of the chest is generally flat irrespective of biological sex of the subject and body size. Accordingly, placement of the example wearable device 300 is easier relative to placement of some conventional wearable devices for monitoring cardiopulmonary condition(s) of a subject. Ease of placement of the example wearable device 300 may improve adherence to a monitoring protocol in subjects with limited dexterity.

In some alternative or additional aspects, as is illustrated in FIG. 3B, the flexible frame 310 includes an inertial sensor device 390 configured to generate measurement data representative of secondary chest sounds. In some alternative or additional aspects, the inertial sensor device 390 is a multiple-axis accelerometer device (e.g., three-axis accelerometer device). In some alternative or additional aspects, the flexible frame 310 has a planar surface 318 (FIG. 3B) having attached thereto an adhesive layer that covers the planar surface. The planar surface 318 excludes respective surfaces of the electrodes 370a-370b, the sensor device 380, and the inertial sensor device 390. Upon placement of the flexible frame 310 on the chest of a subject, the adhesive layer attaches to the chest. In some aspects, a single non-adhesive removable layer overlays the adhesive layer and is removed (e.g., peeled off) before placement of the flexible frame 310 on the chest. In some alternative aspects, multiple non-adhesive removable layers overlay the adhesive layer and are removed (e.g., peeled off) before placement of the flexible frame 310 on the chest. By having an adhesive layer that is exposed entirely by removal of the single non-adhesive removable layer or the multiple non-adhesive removable layers, placement of the example wearable device 300 is significantly simplified relative to conventional monitoring devices. Such a simplification may improve adherence to a monitoring protocol in subjects with limited dexterity.

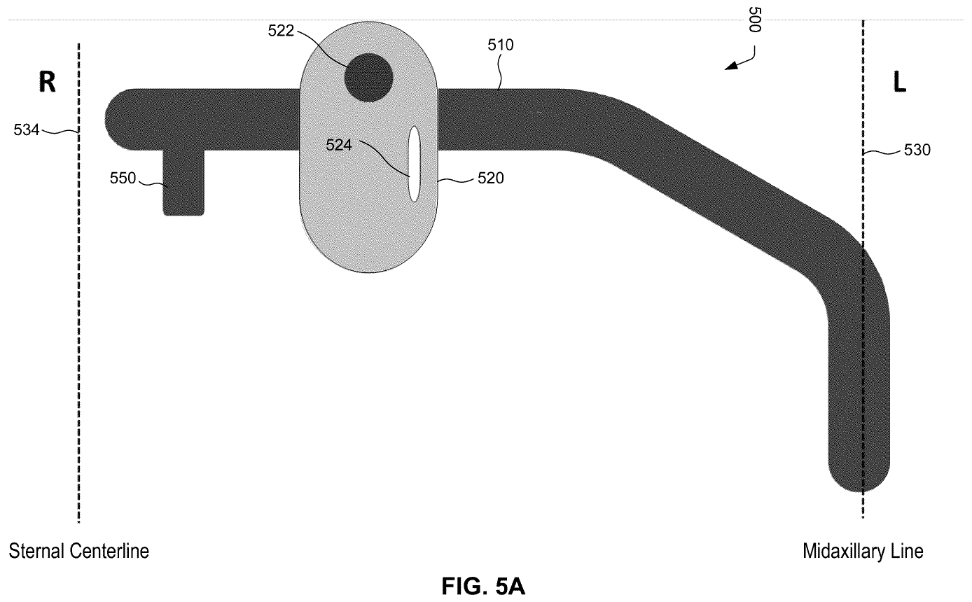

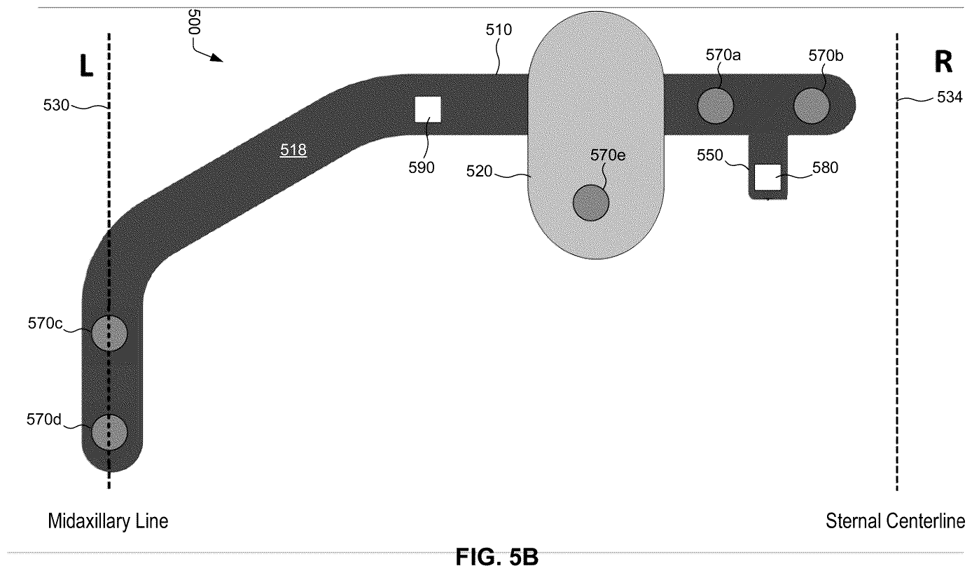

FIG. 5A and FIG. 5B are respective plan views of an example wearable device 500 to monitor health of a subject using multiple measurement modalities, in accordance with one or more aspects of this disclosure. The plan view shown in FIG. 5A is a top view in that the plan view presents the example wearable device 500 as observed from a vantage point located over the chest of the subject and oriented towards an outer surface of the chest of the subject. The plan view shown in FIG. 5A presents the example wearable device 500 as viewed from outside the body of the subject, towards the chest of the subject. The other plan view shown in FIG. 5B is opposite the top view shown in FIG. 5A. The plan view shown in FIG. 5B is a bottom view in that the plan view presents the example wearable device 500 as viewed from a surface of the chest of the subject, towards the exterior of the body of the subject. In some alternative or additional aspects, the bottom view is skin side, i.e., presents the side of a wearable device in accordance with this disclosure that contacts the skin of a subject wearing the wearable device (e.g., the example wearable device 500) in the prescribed placement described herein. In one non-limiting example, the example wearable device 500 embodies or forms part of the wearable device 110 (FIG. 1).

With reference to FIG. 5A, the example wearable device 500 includes a flexible frame 510. In some alternative or additional aspects, the example wearable device 500 includes a housing unit 520. The housing unit 520 contains one or more of the processors 122, the memory devices 126, the I/O interfaces 130, the network adapter(s) 134, or the electric sensor devices 120a. In some alternative or additional aspects, the housing unit 520 includes a button 522 or another interface element that energizes and/or initiates a measurement using the example wearable device 500. In some alternative or additional aspects, the housing unit 520 includes one or more light source devices, one or more speakers, and/or other types of I/O elements 524 that are part of the I/O interfaces 130. The flexible frame 510 is configured to be mounted to the chest of a subject according to a defined placement. In some alternative or additional aspects, the flexible frame 510 is a monolithic structure having a defined non-adjustable size, i.e., the flexible frame 510 is a one-size-fits-all element. The example wearable device 500 need not be sized to the subject being monitored. For example, a clinician or another caregiver does not need to size the example wearable device 500 to a particular subject being monitored before the example wearable device 500 is used to monitor a medical condition of that particular subject.

The disclosure is not limited to the flexible frame 510 being a monolithic structure. In some alternative or additional aspects, the flexible frame 510 is assembled from various separate members having appropriate shape and size so as to provide a one-size-fits-all structure in accordance with aspects of this disclosure.

In the defined placement, the flexible frame 510 is placed on the left side of the chest of the subject, i.e., the flexible frame 510 is placed to the left of the sternal centerline 534 of the subject, spatially separated from the sternum (not depicted in FIG. 5A) of the subject, towards the left midaxillary line 530 of the subject. As such, in the defined placement, the flexible frame 510 reduces or avoids contact with skin that overlays the sternum. In some alternative or additional aspects, in the defined placement, the flexible frame 310 at least partially surrounds breast tissue of the subject from above (i.e., from a region proximate the left clavicle of the subject, where the region is located on the subject). In the defined placement, the wearable device 500 is placed on the subject between the sternal centerline 534 and the left midaxillary line 530. In one non-limiting example, the subject is the subject 210 (FIG. 2).

The flexible frame 510 is elongated and extends from a first end to a second end along a path having a first bend and a second bend. More specifically, the flexible frame 510 includes a first elongated section having the first end, where in the defined placement, the first end is positioned adjacent the sternal centerline 534 and the first elongated section extends towards the left midaxillary line 530. The flexible frame 510 also includes a second elongated section having the second end, with the second elongated section being spaced apart from the first elongated section. In the defined placement, the second elongated section extends along the left midaxillary line 530.

In some alternative or additional aspects, the flexible frame 510 includes an extension member 550 integrated with the flexible frame 510. The extension member 550 extends from a portion of the first elongated section. In the defined placement, the extension member 550 is positioned adjacent the sternum (not depicted in FIG. 5A) of a subject, and at least a portion of the extension member 550 covers an area within a thoracic cavity (chest cavity) of the subject. In one non-limiting example, the portion of the extension member 550 covers an area on a left side of the thoracic cavity. In one non-limiting example, the portion of the extension member covers an area adjacent or over a heart of the subject that is within the thoracic cavity. In some alternative or additional aspects, the extension member 550 is smaller than the extension member 350 in the example wearable device 300 (FIG. 3A and FIG. 3B). The example wearable device 500 has a form factor that is smaller than the form factor of the example wearable device 300.

The example wearable device 500 includes multiple electrodes, including a first electrode 570a, a second electrode 570b, a third electrode 570c, and a fourth electrode 570d. In some alternative or additional aspects, the multiple electrodes include a fifth electrode 570e. As is illustrated in FIG. 5B, the first elongated section has integrated therein or attached thereto a first pair of electrodes consisting of the first electrode 570a and the second electrode 570b. Further, the second elongated section has integrated therein or otherwise attached thereto a second pair of electrodes consisting of the third electrode 570c and the fourth electrode 570d.

In some alternative or additional aspects, the first pair of electrodes and the second pair of electrodes are used for measurements of thoracic impedance in a four-point configuration. An electrode in each of the first pair and the second pair of electrodes is configured as a force electrode, and another electrode in each of the first pair and the second pair of electrodes is configured as a sense electrode. In one non-limiting example, the first electrode 570a and the second electrode 570b are configured as a first sense electrode and a first force electrode, respectively, and the third electrode 570c and the fourth electrode 570d are configured as a second sense electrode and a second force electrode, respectively.

In some aspects, the first force electrode is configured to inject current into the chest of a subject (e.g., subject 210 (FIG. 2)) and the second force electrode is configured to receive the current injected into the chest of the subject, i.e., the first force electrode and the second force electrode force circulation of the injected current through the chest of the subject. In some alternative or additional aspects, the first force electrode is a positive terminal “F+” and the second force electrode is a negative terminal “F−.” In some alternative or additional aspects, the first sense electrode and the second sense electrode are configured to sense a voltage difference caused by the current injected into the chest of the subject. In some alternative or additional aspects, the first sense electrode is a positive terminal “S+” and the second sense electrode is a negative terminal “S−.” The measured thoracic impedance is based on the voltage difference and the amount of current injected into the chest of the torso of the subject.

In some alternative or additional aspects, in addition to measurement data indicative of thoracic impedance of a subject, the example wearable device 500 permits generating measurement data indicative of electrical activity of a subject, as part of generating an ECG. The fifth electrode 570e serves as an RLD electrode and a pair of the first electrode 570a, the second electrode 570b, the third electrode 570c, or the fourth electrode 570d are configured as a limb lead that is similar to Lead II (one of the six limb leads in a standard 12-lead ECG). In one non-limiting example, one of the following combinations of electrodes is configured as Lead II: the first electrode 570a and the third electrode 570c; the first electrode 570a and the fourth electrode 570d; the second electrode 570b and the third electrode 570c; or the second electrode 570b and the fourth electrode 570d.

In some alternative or additional aspects, in the defined placement of the example wearable device 500 onto the chest of a subject (e.g., subject 210 (FIG. 2)), in accordance with aspects described herein, measurement data representative of electrical activity permits detecting P-waves in ECG. Consequently, the example wearable device 500 is used to monitor COPD of a subject, thus detecting COPD or trends in the health status of the subject that may be indicative of the subject being at risk of COPD or progressing towards onset of COPD.

In some alternative or additional aspects, as is illustrated in FIG. 5B, the extension member 550 has integrated therein or attached thereto a sensor device 580 configured to generate measurement data representative of chest sounds. At least a portion of the extension member 550 covers an area within a thoracic cavity (chest cavity) of the subject with the sensor device 580 abutting at least a portion of the area. In some alternative or additional aspects, the sensor device 580 is a MEMS microphone. Because a MEMS microphone is smaller than a piezoelectric microphone, integration of the MEMS microphone into the example device 500 permits reducing and/or or simplifying a form factor of the example device 500 relative to wearable devices that include the piezoelectric microphone. Indeed, by using a MEMS microphone, the extension member 550 is smaller than the extension member 350 in the example wearable device 300 (FIG. 3A and FIG. 3B). As a result, the form factor of the example wearable device 500 is smaller than the form factor of the example wearable device 300.

In some alternative or additional aspects, as is illustrated in FIG. 5B, the flexible frame 510 includes an inertial sensor device 590 configured to generate measurement data representative of second chest sounds. In some alternative or additional aspects, the inertial sensor device 590 is a multiple-axis accelerometer device (e.g., three-axis accelerometer device). In some alternative or additional aspects, the flexible frame 510 has a planar surface 518 (FIG. 5B) having attached thereto an adhesive layer that covers the planar surface. In some alternative or additional aspects, the planar surface 518 excludes respective surfaces of the electrodes 570a-570b, the sensor device 580, and the inertial sensor device 590. Upon placement of the flexible frame 510 on the chest of a subject, the adhesive layer attaches to the chest. In some aspects, a single non-adhesive removable layer overlays the adhesive layer and is removed (e.g., peeled off) before placement of the flexible frame 510 on the chest. In some alternative aspects, multiple non-adhesive removable layers overlay the adhesive layer and are removed (e.g., peeled off) before placement of the flexible frame 510 on the chest. By having an adhesive layer that is exposed entirely by removal of the single non-adhesive removable layer or the multiple non-adhesive removable layers, placement of the example wearable device 500 is significantly simplified relative to conventional monitoring devices. Such a simplification may improve adherence to a monitoring protocol in subjects with limited dexterity.

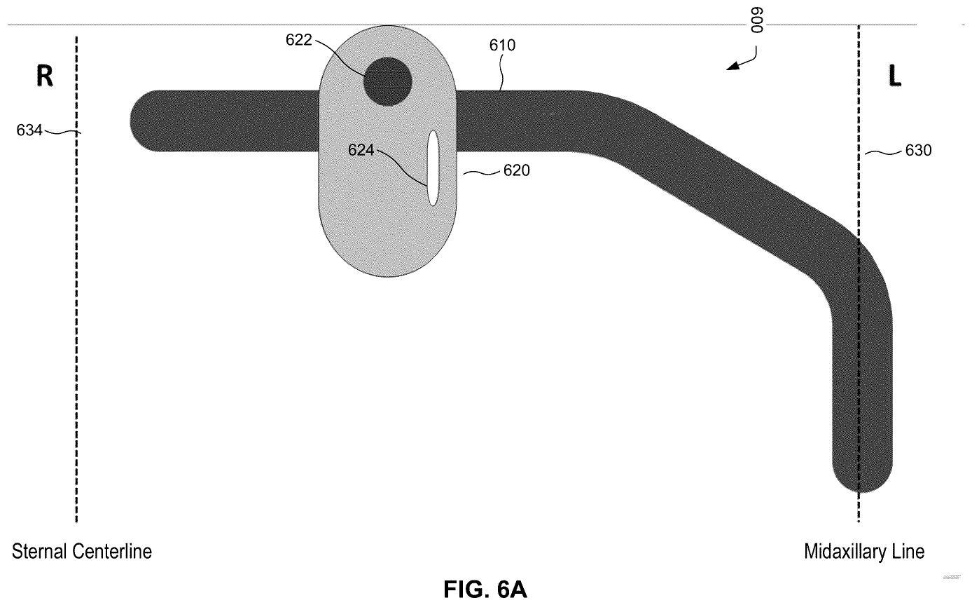

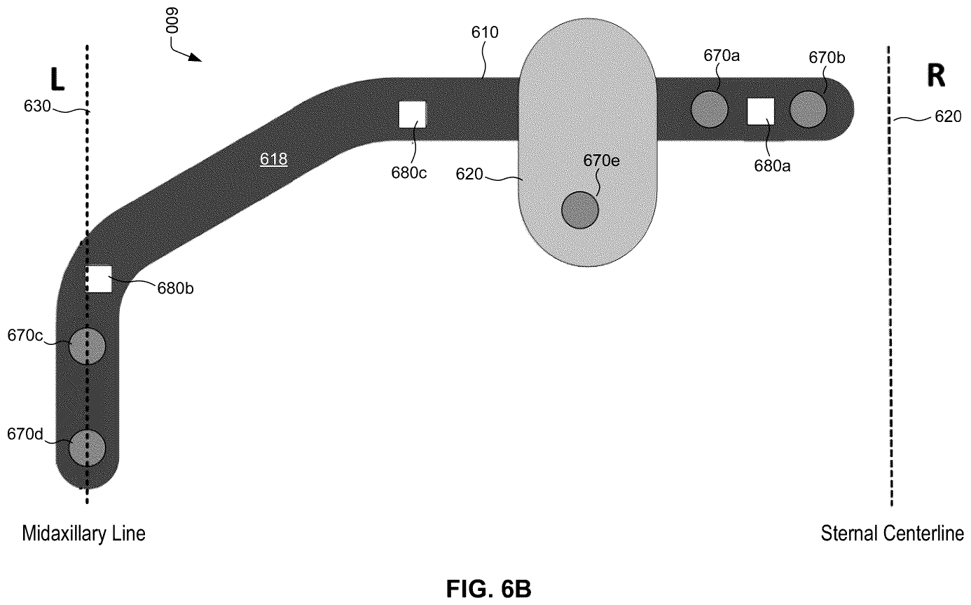

FIG. 6A and FIG. 6B are respective plan views of an example wearable device 600 to monitor health of a subject using multiple measurement modalities, in accordance with one or more aspects of this disclosure. The plan view shown in FIG. 6A is a top view in that the plan view presents the example wearable device 600 as observed from a vantage point located over the chest of the subject and oriented towards an outer surface of the chest of the subject. The plan view shown in FIG. 6A presents the example wearable device 600 as viewed from outside the body of the subject, towards the chest of the subject. The other plan view shown in FIG. 6B is opposite the top view shown in FIG. 6A. The plan view shown in FIG. 6B is a bottom view in that the plan view presents the example wearable device 600 as viewed from a surface of the chest of the subject, towards the exterior of the body of the subject. In some alternative or additional aspects, the bottom is skin side, i.e., the bottom view presents the side of a wearable device in accordance with this disclosure that contacts the skin of a subject wearing the wearable device (e.g., the example wearable device 600) in the prescribed placement described herein. In one non-limiting example, the example wearable device 600 embodies or forms part of the wearable device 110 (FIG. 1).

With reference to FIG. 6A, the example wearable device 600 includes a flexible frame 610. In some alternative or additional aspects, the example wearable device 600 includes a housing unit 620. The housing unit 620 contains one or more of the processors 122, the memory devices 126, the I/O interfaces 130, the network adapter(s) 134, or the electric sensor devices 120a. In some alternative or additional aspects, the housing unit 620 includes a button 622 or another interface element that energizes and/or initiates a measurement using the example wearable device 600. In some alternative or additional aspects, the housing unit 620 includes one or more light source devices, one or more speakers, and/or other types of I/O elements 624 that are part of the I/O interfaces 130. The flexible frame 610 is configured to be mounted to the chest of a subject according to a defined placement. In some alternative or additional aspects, the flexible frame 610 is a monolithic structure having a defined non-adjustable size, i.e., the flexible frame 610 is a one-size-fits-all element. As a result, the example wearable device 600 need not be sized to the subject being monitored. For example, a clinician or another caregiver does not need to size the example wearable device 600 to the subject before the example wearable device 600 is used to monitor a medical condition of the subject.

The disclosure is not limited to the flexible frame 610 being a monolithic structure. In some alternative or additional aspects, the flexible frame 610 is assembled from various separate members having appropriate shape and size so as to provide a one-size-fits-all structure in accordance with aspects of this disclosure.

In the defined placement, the flexible frame 610 is placed on the left side of the chest of the subject, i.e., the flexible frame 610 is placed to the left of the sternal centerline 634 of the subject, spatially separated from the sternum (not depicted in FIG. 6A) of the subject, towards the left midaxillary line 630 of the subject. As such, in the defined placement, the flexible frame 610 reduces or avoids contact with skin that overlays the sternum. In some alternative or additional aspects, in the defined placement, the flexible frame 610 at least partially surrounds breast tissue of the subject from above, i.e., from a region proximate the left clavicle of the subject, where the region is located on the subject. In the defined placement, the wearable device 600 is placed on the subject between the sternal centerline 634 and the left midaxillary line 630. In one non-limiting example, the subject is the subject 210 (FIG. 2).

The flexible frame 610 is elongated and extends from a first end to a second end along a path having a first bend and a second bend. More specifically, the flexible frame 610 includes a first elongated section having the first end, where in the defined placement, the first end is positioned adjacent the sternal centerline 634 and the first elongated section extends towards the left midaxillary line 630. The flexible frame 610 also includes a second elongated section having the second end, with the second elongated section being spaced apart from the first elongated section. In the defined placement, the second elongated section extends along the left midaxillary line 630.

The example wearable device 600 includes multiple electrodes, including a first electrode 670a, a second electrode 670b, a third electrode 670c, and a fourth electrode 670d. In some alternative or additional aspects, the multiple electrodes include a fifth electrode 670e. As is illustrated in FIG. 6B, the first elongated section has integrated therein or attached thereto a first pair of electrodes consisting of the first electrode 670a and the second electrode 670b. Further, the second elongated section has integrated therein or otherwise attached thereto a second pair of electrodes consisting of the third electrode 670c and the fourth electrode 670d. In some alternative or additional aspects, the first pair of electrodes and the second pair of electrodes are used for measurements of thoracic impedance in a four-point configuration. An electrode in each of the first pair and the second pair of electrodes is configured as a force electrode, and another electrode in each of the first pair and the second pair of electrodes is configured as a sense electrode. In one non-limiting example, the first electrode 670a and the second electrode 670b are configured as a first sense electrode and a first force electrode, respectively, and the third electrode 670c and the fourth electrode 670d are configured as a second sense electrode and a second force electrode, respectively.

In some aspects, the first force electrode is configured to inject current into the chest of a subject (e.g., subject 210 (FIG. 2)) and the second force electrode is configured to receive the current injected into the chest of the subject, i.e., the first force electrode and the second force electrode force circulation of the injected current through the chest of the subject. In some alternative or additional aspects, the first force electrode is a positive terminal “F+” and the second force electrode is a negative terminal “F−.” In some alternative or additional aspects, the first sense electrode and the second sense electrode are configured to sense a voltage difference caused by the current injected into the chest of the subject. In some alternative or additional aspects, the first sense electrode is a positive terminal “S+” and the second sense electrode is a negative terminal “S−.” The measured thoracic impedance is based on the voltage difference and the amount of current injected into the chest of the torso of the subject.

In some alternative or additional aspects, in addition to measurement data indicative of thoracic impedance of a subject, the example wearable device 600 permits generating measurement data indicative of electrical activity of a subject, as part of generating an ECG. The fifth electrode 670e serves as a right-left drive (RLD) electrode and a pair of the first electrode 670a, the second electrode 670b, the third electrode 670c, or the fourth electrode 670d are configured as a limb lead that is similar to Lead II (one of the six limb leads in a standard 12-lead ECG). In one non-limiting example, one of the following combinations of electrodes is configured as Lead II: the first electrode 670a and the third electrode 670c; the first electrode 670a and the fourth electrode 670d; the second electrode 670b and the third electrode 670c; and the second electrode 670b and the fourth electrode 670d.

In some alternative or additional aspects, in the defined placement of the example wearable device 600 onto a chest of a subject (e.g., subject 210 (FIG. 2)), in accordance with aspects described herein, measurement data representative of electrical activity permits detecting P-waves in ECG. Consequently, the example wearable device 600 is used to monitor COPD of a subject, thus detecting COPD or trends in the health status of the subject that may be indicative of the subject being at risk of COPD or progressing towards onset of COPD.

In some alternative or additional aspects, as is also illustrated in FIG. 6B, the example wearable device 600 includes multiple inertial sensor devices. Specifically, the first elongated section of flexible frame 610 has integrated therein or attached thereto a first inertial sensor device 680a configured to generate measurement data representative of first chest sounds. In some aspects, the second bend of the flexible frame 610 has integrated therein or attached thereto a second inertial sensor device 680b. In some alternative or additional aspects, the second elongated section of the flexible frame 610 has integrated therein or attached thereto the second inertial sensor device 680b. Regardless of location, the second inertial sensor device 680b is configured to generate measurement data representative of second chest sounds. In some alternative or additional aspects, the flexible frame 610 has integrated therein or attached thereto a third inertial sensor device 680c configured to generate measurement data representative of third chest sounds. In one non-limiting example, the first inertial sensor device 680a is a multiple-axis (e.g., three-axis) accelerometer device, the second inertial sensor device 680b is another multiple-axis (e.g., three-axis) accelerometer device, and the third inertial sensor device 680c is yet another multiple-axis (e.g., three-axis) accelerometer device.

In some alternative or additional aspects, spatial distribution of the multiple inertial sensor devices on the flexible frame 610 is directed to detecting chest vibrations originating mainly from different locations in the chest of a subject. Chest vibrations detected by a particular inertial sensor device of the multiple inertial sensor devices include contributions from motion caused by various physiological phenomena. In some alternative or additional aspects, these contributions include a first type of contributions and a second type of contributions. The first type of contributions originate from movement of the chest wall, circulation of blood outside the heart of the subject and/or other physiological phenomena besides lung sounds and heart sounds. In some alternative or additional aspects, the first type of contributions are similar—in magnitude and/or frequency, for example—across the multiple inertial sensor devices. The first type of contributions are common-mode contributions.

The second type of contributions detected by the particular inertial sensor device originate from heart sounds or lung sounds, or both. Heart sounds and lung sounds permit monitoring a medical condition of a subject. In some cases, the medical condition is a cardiopulmonary condition, e.g., CHF or COPD. A location of the particular inertial sensor device on the flexible frame 610 determine if second type of contributions originate from heart sounds or lung sounds. In one non-limiting example, location of the first inertial sensor device 680a in the flexible frame 610 causes, in the defined placement described herein, the first inertial sensor device 680a to be proximate to the heart. Consequently, in the defined placement, the first inertial sensor device 680a generates measurement data representative of chest vibrations including common-mode contributions and other contributions (i.e., the second type of contribution) originating from heart sounds. The heart sounds include systolic sounds and diastolic sounds. Systolic sounds include S1 sounds, S2 sounds, and diastolic murmurs. S1 sounds and S2 sounds are normal (unless a wide split in S2 sounds is present). Diastolic sounds include diastolic murmurs, and S3 sounds and S4 sounds that originate in heart ventricles. Presence of S3 sounds and S4 sounds, individually or in any combination, are pathological and may indicate presence of CHF. In one non-limiting example, location of the third inertial sensor device 680c in the flexible frame 610 causes, in the defined placement described herein, the third inertial sensor device 680c to be proximate to a lung of the subject. Consequently, in the defined placement, the third inertial sensor device 680c can generate measurement data representative of chest vibrations including common-mode contributions and other contributions (i.e., the second type of contribution) originating from lung sounds.

This disclosure is not limited to inertial sensor devices. In some non-limiting examples, instead of multiple inertial sensor devices, the flexible frame 610 has integrated therein or attached thereto one or more multiple MEMS microphone devices (not depicted in FIG. 6B) that replace respective ones of the inertial sensor devices 680a-680c. Each of the MEMS microphone devices provide the same or similar functionality as one of the inertial sensor devices 680a-680c.

In some alternative or additional aspects, one or more processors 122 included in the housing unit 620 or functionally coupled (e.g., communicatively coupled) to the example wearable device 600 operates on measurement datasets (or groups of signals) generated by respective ones of the first inertial sensor device 680a, the second inertial sensor device 680b, and the third inertial sensor device 680c. By operating on such measurement datasets, the processors 122 can remove common-mode contributions and permit or otherwise facilitate distinguishing between the heart sounds and lung sounds specific to different regions of the thoracic cavity of a subject. Spatial separation (i.e., spatial distribution) of the inertial sensor devices 680a-680c permits better differentiation between a correlated signal and uncorrelated noise. The uncorrelated noise, which is random and varies independently across different locations of the inertial sensor devices 680a-680c, is more effectively distinguished and filtered out. As a result, an overall signal-to-noise ratio (SNR) is improved, leading to higher quality signal detection and processing. In some alternative or additional aspects, operating on the measurement datasets (or groups of signals) includes finding a cross-correlation using principal component analysis (PCA) or singular value decomposition (SVD).

In some alternative or additional aspects, the flexible frame 610 has a planar surface 618 (FIG. 6B) that is attached thereto an adhesive layer that covers the planar surface. In some alternative or additional aspects, the planar surface 618 excludes respective surfaces of the electrodes 670a-670b and the inertial sensors 680a-680c. Upon placement of the flexible frame 610 on the chest of a subject, the adhesive layer attaches to the chest. In some aspects, a single non-adhesive removable layer overlays the adhesive layer and is removed (e.g., peeled off) before placement of the flexible frame 610 on the chest. In some alternative aspects, multiple non-adhesive removable layers overlays the adhesive layer and are removed (e.g., peeled off) before placement of the flexible frame 610 on the chest. By having an adhesive layer that is exposed entirely by removal of the single non-adhesive removable layer or the multiple non-adhesive removable layers, placement of the example wearable device 600 is significantly simplified relative to conventional monitoring devices. Such a simplification may improve adherence to a monitoring protocol in subjects with limited dexterity.

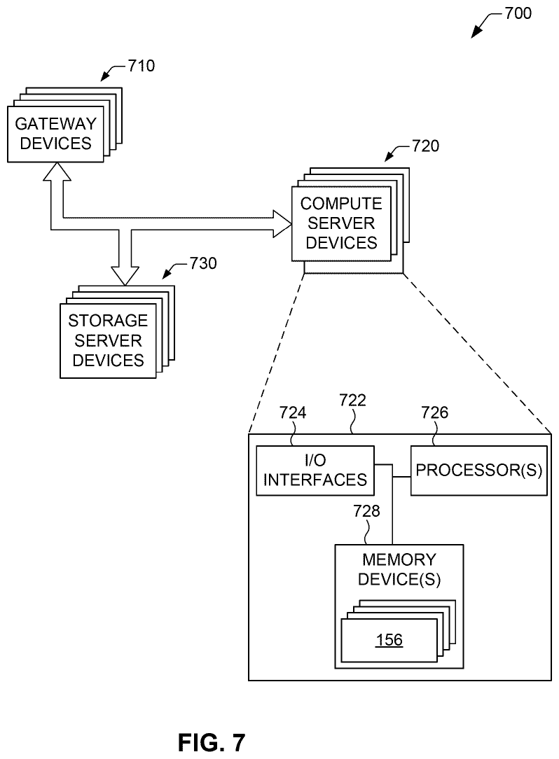

FIG. 7 is a schematic block diagram of an example computing system 700 that implements analysis of multi-modal measurement signals generated by the wearable device 110 (FIG. 1), in accordance with one or more embodiments of this disclosure. In some alternative or additional aspects, the computing system 700 embodies, or is part of, the processing platform 150 described herein in connection with example systems of this disclosure. Accordingly, the example computing system 700 provides at least some of the functionality described herein in connection with monitoring status changes of a cardiopulmonary condition using multi-modal measurement signals in accordance with aspects described herein.

In some alternative or additional aspects, the processing platform 150 includes multiple server devices 154. The multiple server devices 154 are arranged in a cloud architecture. As such, the example computing system 700 includes two types of server devices: compute server devices 720 and storage server devices 730. A subset of the compute server devices 720, individually or in any combination, host various modules that permit implementing the analysis included in monitoring status changes of a cardiopulmonary condition using multi-modal measurement signals in accordance with aspects described herein. The subset of compute server devices 720 operate in accordance with functionality described herein in connection with monitoring status changes of a cardiopulmonary condition using multi-modal measurement signals. The architecture of each of the compute server devices 720, including the compute server device 722, includes multiple input/output (I/O) interfaces 724, one or more processors 726, one or more memory devices 728, and a bus architecture that functionally couples (e.g., electrically coupled, mechanically coupled, and/or communicatively couples) the processors 726 and the memory devices 728. In some alternative or additional aspects, the compute server device 722 stores such modules in at least one of such memory devices 728. At least the subset of the compute server devices 720 is communicatively coupled to one or more of the storage server devices 730. The coupling is direct or is mediated by one or more gateway devices 710. The storage server devices 730 include data and/or metadata that are used to implement the functionality described herein in connection with monitoring status changes of a cardiopulmonary condition using multi-modal measurement signals.

In some alternative or additional aspects, each gateway device 710 includes one or multiple processors functionally coupled (e.g., electrically coupled, mechanically coupled, and/or communicatively coupled) to one or multiple memory devices that retain application programming interfaces (APIs) and/or other types of program code for access to the compute server devices 720 and storage server devices 730. In one non-limiting example, such access is programmatic, via a defined function call. The subset of the compute server devices 720 host one or a combination of modules that use APIs supplied by the gateway devices 710 in order to provide results of implementing the functionalities described herein in connection with monitoring status changes of a cardiopulmonary condition using multi-modal measurement signals in accordance with aspects described herein.

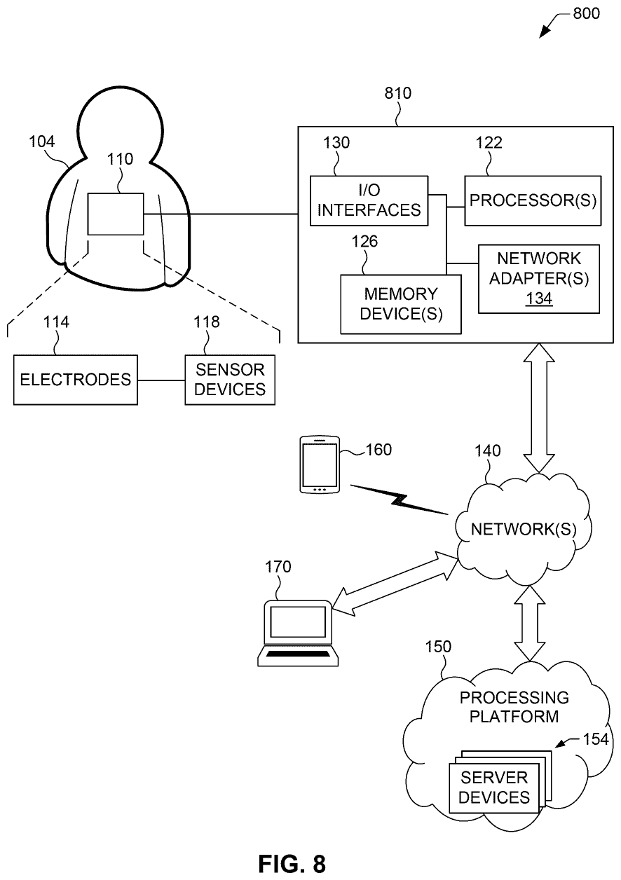

Other architectures of systems to monitor status changes of a cardiopulmonary condition are implemented. As an illustration, FIG. 8 is a schematic block diagram of an example system 800 to monitor status changes of a cardiopulmonary condition that is implemented in accordance with one or more aspects of this disclosure. In the example system 800, the structure of the wearable device 110 is simplified relative to the example system 100 (FIG. 1). In some alternative or additional aspects, a gateway device 810 communicatively coupled with the wearable device 110 provides control functionality for the wearable device 110. In some alternative or additional aspects, the gateway device 810 is communicatively coupled with the processing platform 150, and provides data and signaling to the processing platform 150. The data is indicative of measurement signals generated by the multiple sensor devices 118. In some alternative or additional aspects, the gateway device 810 is communicatively coupled with the user device 160 and the computing device 170 via at least one of the networks 140.

In some alternative or additional aspects, the computing device 170 is located in a healthcare facility or another type of facility (e.g., a group home, a nursing home, etc.). Regardless of its type, the facility or location of the computing device 170 is represented by element 172. In some alternative or additional aspects, the computing device 170 is part of a hospital system of computing devices. In some alternative or additional aspects, the computing device 170 is operated by a clinician responsible for providing care to the subject 104. As such, the computing device 170 is a clinician device. In some alternative or additional aspects, the computing device 170 is configured with a software application, or another type of program code, to exchange information with other devices described herein, and also is configured to present information corresponding or otherwise associated with measurements of the various signals described herein, as performed using the wearable device 110 or other wearable devices in accordance with aspects of this disclosure. In some alternative or additional aspects, the computing device 170 presents at least some of the information in one or more user interfaces 174.

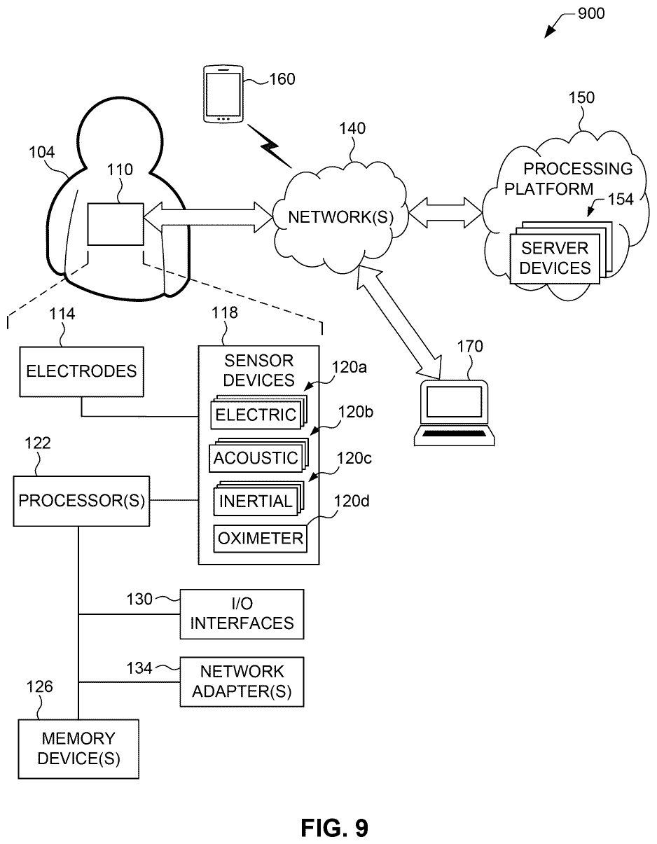

As another illustration, FIG. 9 is a schematic block diagram of another example system 900 to monitor status changes of a cardiopulmonary condition that is implemented in accordance with one or more aspects of this disclosure. In the example system 900, the wearable device 110 is communicatively coupled with the processing platform 150 via at least one of the networks 140, and provides data and signaling to the processing platform 150. The data is indicative of measurement signals generated by the multiple sensor devices 118. Rather than being directly communicatively coupled with the user device 160, as is the case in the example system 100 (FIG. 1), the wearable device 110 is communicatively coupled with the user device 160 via the network(s) 140.

Although the sensor devices 118 have been illustrated and described herein as including numerous types of sensor devices, this disclosure is not limited in that respect. Indeed, in some cases, more, fewer, or other types of sensor devices are present in the sensor devices 118. In some alternative or additional aspects, the memory devices 126 retain processor-accessible instructions (e.g., processor-readable instructions and/or processor-executable instructions) that, in response to being executed by the processor(s), individually or in combination, provide at least some of the functionality of the modules 156.

Various aspects of the disclosure take the form of an entirely or partially hardware aspect, an entirely or partially software aspect, or a combination of software and hardware. Furthermore, as described herein, various aspects of the disclosure (e.g., systems and devices) take the form of a computer program product comprising a computer-readable non-transitory storage medium having processor-accessible instructions (e.g., computer-readable and/or computer-executable instructions) such as computer software, encoded or otherwise embodied in such storage medium. In some alternative or additional aspects, those instructions are read or otherwise accessed and executed by one or more processors, individually or in combination, to perform or permit the performance of the operations described herein. In some alternative or additional aspects, the instructions are provided in any suitable form, such as source code, compiled code, interpreted code, executable code, static code, dynamic code, assembler code, combinations of the foregoing, and the like. Any suitable computer-readable non-transitory storage medium is utilized to form the computer program product. For instance, the computer-readable medium includes any tangible non-transitory medium for storing information in a form readable or otherwise accessible by one or more computers or processor(s) functionally coupled thereto. Non-transitory storage media can include read-only memory (ROM); random access memory (RAM); magnetic disk storage media; optical storage media; flash memory, and so forth.

Aspects of this disclosure are described herein with reference to block diagrams and flowchart illustrations of processor-implemented methods, systems, devices, apparatuses, and computer program products. In some alternative or additional aspects, it is understood that each block of the block diagrams and flowchart illustrations, and combinations of blocks in the block diagrams and flowchart illustrations, respectively, is implemented by processor-accessible instructions. Such instructions include, for example, computer program instructions (e.g., processor-readable and/or processor-executable instructions). The processor-accessible instructions are built (e.g., linked and compiled) and retained in processor-executable form in one or multiple memory devices or one or many other processor-accessible non-transitory storage media. In some alternative or additional aspects, these computer program instructions are stored in a processor-readable memory, where in response to execution by one or more processors, individually or in combination, the computer program instructions can direct a computer, a computing device, or other programmable data processing apparatus to function in a particular manner, such that the instructions stored in the processor-readable memory produce an article of manufacture including processor-accessible instructions (e.g., processor-readable instructions and/or processor-executable instructions) to implement the function specified in the flowchart blocks (individually or in a particular combination) or blocks in block diagrams (individually or in a particular combination). In some alternative or additional aspects, the computer program instructions are loaded onto a computer, a computing device, or other programmable data processing apparatus to cause a series of operations to be performed on the computer or other programmable apparatus to produce a computer-implemented process. The series of operations are performed in response to execution by one or more processor or other types of processing circuitry. Thus, such instructions that execute on the computer or other programmable apparatus provide operations for implementing the functions specified in the flowchart blocks (individually or in a particular combination) or blocks in block diagrams (individually or in a particular combination).

In some alternative or additional aspects, the processor-accessible instructions are loaded or otherwise incorporated into a general-purpose computer, a special purpose computer, or another programmable information processing apparatus to produce a particular machine, such that the operations or functions specified in the flowchart block or blocks are implemented in response to execution at the computer or processing apparatus. More specifically, the loaded processor-accessible instructions are accessed and executed by one or multiple processors, individually or in combination, or other types of processing circuitry. In response to execution, the loaded processor-accessible instructions provide the functionality described in connection with flowchart blocks (individually or in a particular combination) or blocks in block diagrams (individually or in a particular combination). Thus, such instructions which execute on a computer, a computing device, or other programmable data processing apparatus can create a means for implementing the functions specified in the flowchart blocks (individually or in a particular combination) or blocks in block diagrams (individually or in a particular combination).

Unless otherwise expressly stated, it is in no way intended that any protocol, procedure, process, or method set forth herein be construed as requiring that its acts or steps be performed in a specific order. Accordingly, where a process or method claim does not actually recite an order to be followed by its acts or steps or it is not otherwise specifically recited in the claims or descriptions of the subject disclosure that the steps are to be limited to a specific order, it is in no way intended that an order be inferred, in any respect. This holds for any possible non-express basis for interpretation, including: matters of logic with respect to the arrangement of steps or operational flow; plain meaning derived from grammatical organization or punctuation; the number or type of aspects described in the specification or annexed drawings; or the like.

As used in this disclosure, including the annexed drawings, the terms “component,” “module,” “interface,” “system,” and the like are intended to refer to a computer-related entity or an entity related to an apparatus with one or more specific functionalities. The entity is either hardware, a combination of hardware and software, software, or software in execution. One or more of such entities are also referred to as “functional elements.” As an example, a component is a process running on a processor, a processor, an object, an executable, a thread of execution, a program, and/or a computer. For example, both an application running on a server or network controller, and the server or network controller is a component. One or more components can reside within a process and/or thread of execution and a component is localized on one computer and/or distributed between two or more computers. Also, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component is an apparatus with specific functionality provided by mechanical parts operated by electric or electronic circuitry, which parts is controlled or otherwise operated by program code executed by a processor. As yet another example, a component is an apparatus that provides specific functionality through electronic components without mechanical parts, the electronic components can include a processor to execute program code that provides, at least partially, the functionality of the electronic components. As still another example, interface(s) can include I/O components or Application Programming Interface (API) components. While the foregoing examples are directed to aspects of a component, the exemplified aspects or features also apply to a system, module, and similar.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in this specification and annexed drawings should be construed to mean “one or more” unless specified otherwise or clear from context to be directed to a singular form.

In addition, the terms “example” and “such as” are utilized herein to mean serving as an instance or illustration. Any aspect or design described herein as an “example” or referred to in connection with a “such as” clause is not necessarily to be construed as preferred or advantageous over other aspects or designs described herein. Rather, use of the terms “example” or “such as” is intended to present concepts in a concrete fashion. The terms “first,” “second,” “third,” and so forth, as used in the claims and description, unless otherwise clear by context, is for clarity only and doesn't necessarily indicate or imply any order in time or space.

The term “processor,” as utilized in this disclosure, can refer to any computing processing unit or device comprising processing circuitry that can operate on data and/or signaling. A computing processing unit or device can include, for example, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can include an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. In some cases, processors can exploit nano-scale architectures, such as molecular and quantum-dot based transistors, switches and gates, in order to optimize space usage or enhance performance of user equipment. A processor is also be implemented as a combination of computing processing units.

In addition, terms such as “store,” “data store,” data storage,” “database,” and substantially any other information storage component relevant to operation and functionality of a component, refer to “memory components,” or entities embodied in a “memory” or components comprising the memory. It will be appreciated that the memory components described herein is either volatile memory or nonvolatile memory, or can include both volatile and nonvolatile memory. Moreover, in some alternative or additional aspects, a memory component is removable or affixed to a functional element (e.g., device, server).

Simply as an illustration, nonvolatile memory can include read only memory (ROM), programmable ROM (PROM), electrically programmable ROM (EPROM), electrically erasable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Various aspects described herein is implemented as a method, system, device, apparatus, or article of manufacture using standard programming and/or engineering techniques. In addition, various of the aspects disclosed herein also is implemented by means of program modules or other types of computer program instructions stored in a memory device and executed by a processor, or other combination of hardware and software, or hardware and firmware. Such program modules or computer program instructions is loaded onto a general-purpose computer, a special purpose computer, or another type of programmable data processing apparatus to produce a machine, such that the instructions which execute on the computer or other programmable data processing apparatus create a means for implementing the functionality of disclosed herein.

The term “article of manufacture” as used herein is intended to encompass a computer program accessible from any computer-readable device, carrier, or media. For example, computer readable media can include but are not limited to magnetic storage devices (e.g., hard drive disk, floppy disk, magnetic strips, or similar), optical discs (e.g., compact disc (CD), digital versatile disc (DVD), blu-ray disc (BD), or similar), smart cards, and flash memory devices (e.g., card, stick, key drive, or similar).

The present disclosure can be described in accordance with the following numbered Clauses, which should not be confused with the claims.

Clause 1. A wearable device for multi-modal monitoring of a medical condition, comprising: a flexible frame configured to be mounted to a torso of a subject in accordance with a defined placement, the flexible frame comprising: a first elongated section having a first end and including a first pair of electrodes, wherein, in the defined placement, the first end is positioned adjacent a sternal centerline of the subject, and the first elongated section extends towards a left midaxillary line of the subject; and a second elongated section having a second end and including a second pair of electrodes, wherein the second elongated section is spaced apart from the first elongated section, and wherein, in the defined placement, the second elongated section extends along the left midaxillary line; and an extension member integrated with the flexible frame, the extension member extending from a portion of the first elongated section and including a sensor device configured to generate measurement data representative of chest sounds, wherein, in the defined placement, the extension member is positioned adjacent a sternum of the subject and at least a portion of the extension member covers an area within a thoracic cavity of the subject, with the sensor device abutting at least a portion of the area.

Clause 2. The wearable device of clause 1, wherein the flexible frame is a monolithic structure having a defined non-adjustable size.

Clause 3. The wearable device of any one of the preceding clauses, wherein the sensor device is one of an inertial sensor device, a piezoelectric microphone, or a microelectromechanical system (MEMS) microphone.

Clause 4. The wearable device of any one of the preceding clauses, wherein the flexible frame further comprises a planar surface having attached thereto an adhesive layer that covers the planar surface, and wherein the adhesive layer attaches to the torso when the flexible frame is mounted to the torso.

Clause 5. The wearable device of any one of the preceding clauses, wherein the flexible frame further comprises an inertial sensor device configured to generate additional measurement data representative of additional chest sounds.

Clause 6. The wearable device of any one of the preceding clauses, wherein a first electrode of the first pair of electrodes and a second electrode of the second pair of electrodes are configured as a lead defining an electrocardiogram (ECG) vector, with the lead corresponding to Lead II of standard limb leads for a 12-lead ECG.

Clause 7. The wearable device of any one of the preceding clauses, wherein, in the defined placement, the flexible frame at least partially surrounds breast tissue of the subject when mounted to the torso.

Clause 8. A wearable device for multi-modal monitoring of a medical condition, comprising: a flexible frame configured to be mounted to a torso of a subject according to a defined placement, the flexible frame comprising: a first elongated section having a first end and including a first pair of electrodes and an inertial sensor device configured to generate first measurement data representative of first chest sounds, wherein, in the defined placement, the first end is positioned adjacent a sternal centerline of the subject and the first elongated section extends towards a left midaxillary line of the subject; and a second elongated section having a second end and including a second pair of electrodes and a second inertial sensor device configured to generate second measurement data representative of second chest sounds, wherein the second elongated section is spaced apart from the first elongated section, and wherein, in the defined placement, the second elongated section extends along the left midaxillary line.

Clause 9. The wearable device of clause 8, wherein the flexible frame is a monolithic structure having a defined non-adjustable size.

Clause 10. The wearable device of any one of the preceding clauses, wherein the first inertial sensor device comprises a first multiple-axis accelerometer device, and wherein the second inertial sensor device comprises a second multiple-axis accelerometer device.

Clause 11. The wearable device of any one of the preceding clauses, wherein the flexible frame further comprises a multiple-axis accelerometer device configured to generate third measurement data representative of third chest sounds.

Clause 12. The wearable device of any one of the preceding clauses, wherein the flexible frame further comprises a planar surface having attached thereto an adhesive layer that covers the planar surface, wherein the adhesive layer attaches to a chest of the subject when the flexible frame is mounted to the torso.

Clause 13. The wearable device of any one of the preceding clauses, wherein a first electrode of the first pair of electrodes and a second electrode of the second pair of electrodes are configured as a lead defining an electrocardiogram (ECG) vector, with the lead corresponding to Lead II of standard limb leads for a 12-lead ECG.

Clause 14. The wearable device of any one of the preceding clauses, wherein, in the defined placement, the flexible frame at least partially surrounds breast tissue of the subject when mounted to the torso.

Clause 15. A system for multi-modal monitoring of a medical condition, comprising: a flexible frame configured to be mounted to a torso of a subject in accordance with a defined placement, the flexible frame comprising a first elongated section and a second elongated section spaced apart from the first elongated section, wherein each elongated section has a corresponding end and includes a corresponding pair of electrodes; at least one sensor device attached to the flexible frame; one or more memories storing instructions therein; and one or more processors communicatively coupled with the one or more memories and configured, individually or in any combination, to: initiate a measurement of thoracic impedance of the subject via the at least one sensor device; receive, from the at least one sensor device, measurement data representative of chest sounds; and exchange one or more measurement signals based on the measurement data with another device; wherein, in the defined placement, the first elongated section extends towards a left midaxillary line of the subject and a corresponding end the first elongated section is positioned adjacent a sternal centerline of the subject, and the second elongated section extends along the left midaxillary line.

Clause 16. The system of any one of the preceding clauses, wherein the another device is one of a computing device, a user device, or a server device, and wherein the flexible frame is a monolithic structure having a defined non-adjustable size.

Clause 17. The system of any one of the preceding clauses, wherein the at least one sensor device comprises a first sensor device included in an extension member extending from a portion of the first elongated section, and wherein, in the defined placement, the extension member is positioned adjacent a sternum of the subject and at least a portion of the extension member covers an area within a thoracic cavity of the subject, with first the sensor device abutting at least a portion of the area.

Clause 18. The system of any one of the preceding clauses, wherein the at least one sensor device comprises a first sensor device and a second sensor device included in the first elongated section and the second elongated section, respectively, and wherein each sensor device comprises a multiple-axis accelerometer device.

Clause 19. The system of any one of the preceding clauses, wherein a first electrode of a corresponding pair of electrodes of the first elongated section and a second electrode of a corresponding pair of electrodes of the second elongated section are configured as a lead defining an electrocardiogram (ECG) vector, with the lead corresponding to Lead II of standard limb leads for a 12-lead ECG.

Clause 20. The system of any one of the preceding clauses, wherein, in the defined placement, the flexible frame at least partially surrounds breast tissue of the subject when mounted to the torso.

What has been described above includes examples of one or more aspects of the disclosure. It is, of course, not possible to describe every conceivable combination of components or methodologies for purposes of describing these examples, and it is recognized that many further combinations and permutations of the present aspects are possible. Accordingly, the aspects disclosed and/or claimed herein are intended to embrace all such alterations, modifications and variations that fall within the spirit and scope of the detailed description and the appended claims. Furthermore, to the extent that one or more of the terms “includes,” “including,” “has,” “have,” or “having” is used in either the detailed description or the claims, such term is intended to be inclusive in a manner similar to the term “comprising” as “comprising” is interpreted when employed as a transitional word in a claim.

1. A wearable device for multi-modal monitoring of a medical condition, comprising:
a flexible frame configured to be mounted to a torso of a subject in accordance with a defined placement, the flexible frame comprising:
a first elongated section having a first end and including a first pair of electrodes, wherein, in the defined placement, the first end is positioned adjacent a sternal centerline of the subject, and the first elongated section extends towards a left midaxillary line of the subject; and
a second elongated section having a second end and including a second pair of electrodes, wherein the second elongated section is spaced apart from the first elongated section, and wherein, in the defined placement, the second elongated section extends along the left midaxillary line; and
an extension member integrated with the flexible frame, the extension member extending from a portion of the first elongated section and including a sensor device configured to generate measurement data representative of chest sounds, wherein, in the defined placement, the extension member is positioned adjacent a sternum of the subject and at least a portion of the extension member covers an area within a thoracic cavity of the subject, with the sensor device abutting at least a portion of the area.

2. The wearable device of claim 1, wherein the flexible frame is a monolithic structure having a defined non-adjustable size.

3. The wearable device of claim 1, wherein the sensor device is one of an inertial sensor device, a piezoelectric microphone, or a microelectromechanical system (MEMS) microphone.

4. The wearable device of claim 1, wherein the flexible frame further comprises a planar surface having attached thereto an adhesive layer that covers the planar surface, and wherein the adhesive layer attaches to the torso when the flexible frame is mounted to the torso.

5. The wearable device of claim 1, wherein the flexible frame further comprises an inertial sensor device configured to generate additional measurement data representative of additional chest sounds.

6. The wearable device of claim 1, wherein a first electrode of the first pair of electrodes and a second electrode of the second pair of electrodes are configured as a lead defining an electrocardiogram (ECG) vector, with the lead corresponding to Lead II of standard limb leads for a 12-lead ECG.

7. The wearable device of claim 1, wherein, in the defined placement, the flexible frame at least partially surrounds breast tissue of the subject when mounted to the torso.

8. A wearable device for multi-modal monitoring of a medical condition, comprising:
a flexible frame configured to be mounted to a torso of a subject according to a defined placement, the flexible frame comprising:
a first elongated section having a first end and including a first pair of electrodes and an inertial sensor device configured to generate first measurement data representative of first chest sounds, wherein, in the defined placement, the first end is positioned adjacent a sternal centerline of the subject and the first elongated section extends towards a left midaxillary line of the subject; and
a second elongated section having a second end and including a second pair of electrodes and a second inertial sensor device configured to generate second measurement data representative of second chest sounds, wherein the second elongated section is spaced apart from the first elongated section, and wherein, in the defined placement, the second elongated section extends along the left midaxillary line.

9. The wearable device of claim 8, wherein the flexible frame is a monolithic structure having a defined non-adjustable size.

10. The wearable device of claim 8, wherein the first inertial sensor device comprises a first multiple-axis accelerometer device, and wherein the second inertial sensor device comprises a second multiple-axis accelerometer device.

11. The wearable device of claim 8, wherein the flexible frame further comprises a multiple-axis accelerometer device configured to generate third measurement data representative of third chest sounds.

12. The wearable device of claim 8, wherein the flexible frame further comprises a planar surface having attached thereto an adhesive layer that covers the planar surface, wherein the adhesive layer attaches to a chest of the subject when the flexible frame is mounted to the torso.

13. The wearable device of claim 8, wherein a first electrode of the first pair of electrodes and a second electrode of the second pair of electrodes are configured as a lead defining an electrocardiogram (ECG) vector, with the lead corresponding to Lead II of standard limb leads for a 12-lead ECG.

14. The wearable device of claim 8, wherein, in the defined placement, the flexible frame at least partially surrounds breast tissue of the subject when mounted to the torso.

15. A system for multi-modal monitoring of a medical condition, comprising:
a flexible frame configured to be mounted to a torso of a subject in accordance with a defined placement, the flexible frame comprising a first elongated section and a second elongated section spaced apart from the first elongated section, wherein each elongated section has a corresponding end and includes a corresponding pair of electrodes;
at least one sensor device attached to the flexible frame;
one or more memories storing instructions therein; and
one or more processors communicatively coupled with the one or more memories and configured, individually or in any combination, to:
initiate a measurement of thoracic impedance of the subject via the at least one sensor device;
receive, from the at least one sensor device, measurement data representative of chest sounds; and
exchange one or more measurement signals based on the measurement data with another device;
wherein, in the defined placement, the first elongated section extends towards a left midaxillary line of the subject and a corresponding end the first elongated section is positioned adjacent a sternal centerline of the subject, and the second elongated section extends along the left midaxillary line.

16. The system of claim 15, wherein the another device is one of a computing device, a user device, or a server device, and wherein the flexible frame is a monolithic structure having a defined non-adjustable size.

17. The system of claim 15, wherein the at least one sensor device comprises a first sensor device included in an extension member extending from a portion of the first elongated section, and wherein, in the defined placement, the extension member is positioned adjacent a sternum of the subject and at least a portion of the extension member covers an area within a thoracic cavity of the subject, with first the sensor device abutting at least a portion of the area.

18. The system of claim 15, wherein the at least one sensor device comprises a first sensor device and a second sensor device included in the first elongated section and the second elongated section, respectively, and wherein each sensor device comprises a multiple-axis accelerometer device.

19. The system of claim 15, wherein a first electrode of a corresponding pair of electrodes of the first elongated section and a second electrode of a corresponding pair of electrodes of the second elongated section are configured as a lead defining an electrocardiogram (ECG) vector, with the lead corresponding to Lead II of standard limb leads for a 12-lead ECG.

20. The system of claim 15, wherein, in the defined placement, the flexible frame at least partially surrounds breast tissue of the subject when mounted to the torso.
